SEQUENCE LISTING (16 sequences)

SEQ ID NO: 1 - DNA, 22 bp, Artificial Sequence
  misc_feature (1)..(22): Oligonucleotide primer
aggcggaagaactgctgcaacg

SEQ ID NO: 2 - DNA, 22 bp, Artificial Sequence
  misc_feature (1)..(22): Oligonucleotide primer
aggtccatttcgtcgcagaggc

SEQ ID NO: 3 - DNA, 24 bp, Artificial Sequence
  misc_feature (1)..(24): Oligonucleotide primer
atgtttggcaagtaggaagggacc

SEQ ID NO: 4 - DNA, 24 bp, Artificial Sequence
  misc_feature (1)..(24): Oligonucleotide primer
gcattccataagccgtcacgattc

SEQ ID NO: 5 - DNA, 1919 bp, Glycine max
gcacgagaccccacacagcaaccagagcagctatgctagtgagaatcaacacactcctccaaggctactcaggaatcaggtttgaaattttggaggcaatcacaaagcttctgaacaacaacattaccccatgtttgccacttaggggaacaatcacagcatctggtgatcttgttcctttgtcctacattgctggtttgctaactggtagaccaaactccaaggctgttggaccctctggtgagattctgaatgccaaagaagcctttgaattggccaacatcggtgctgagttctttgagttgcaacctaaggaaggccttgcccttgtgaatggcactgctgttggttctggcttggcctcaattgttctatttgaagccaacatcattgctgtcttgtctgaagttatttcagcaatttttgctgaagttatgcaaggaaagcctgaattcactgaccatttgactcataaactaaagcaccaccctggtcagattgaagctgctgctattatggaacacattttggaaggaagctcttacgtgaaagctgctaagaagttgcatgagattgatcctttacaaaagcctaaacaggaccgttatgctcttaggacttcaccacaatggcttggtcctctaattgaagtgattagattctctaccaagtcaattgagagggagattaactcagtcaatgacaaccctttgattgatgtgtcaaggaacaaggcacttcatggtggtaacttccaaggaactcctattggagtctccatggataatacacgtttggctcttgcttcaattggtaaactcatgtttgctcaattctctgagcttgtcaatgattattacaacaatggtttgccttcaaatctcactgccagcagaaaccccagcttggattatggattcaagggagctgaaattgccatggcatcttattgttctgaacttcaatatttggcgaatccggtgacaagccacgtgcaaagcgcggagcaacacaaccaagatgtgaactctctggggctgatttcatcaaggaagactcatgaggctattgagatcctcaagctcatgtcctccactttcctggtcgccctttgccaagccattgacttgaggcatttggaggagaatttgaagaacacggtcaagaacgttgtgagtcaagttgctaagaggactctcaccacaggtgtcaatggagagcttcacccttcaaggttttgtgagaaggacttgctcaaggttgttgatagggagtacacatttgcatacattgatgacccctgcagtggaacataccctttgatgcaaaagctaaggcaagtgcttgtggactatgcattggccaatggagagaacgagaagaacacaaacacatcaatcttccaaaagattgcaacatttgaggaagagttgaagacccttttgcctaaggaagtggaaggtgcaagagttgcatatgagaatgaccaatgtgcaattccaaacaagatcaaggaatgcaggtcttaccccttgtacaagtttgtgagagaggagttggggacagcattgctaactggtgaaagggttatctcaccgggtgaagagtgtgacaaagtgttcactgctttgtgccaagggaagatcattgatccacttttggaatgccttggggagtggaatggggcacctcttccaatatgttagtttttcttattttctgttttcttgaagagtggtttcttttctgtacacgtgtttgtgttgatattaagcatttggtttgtctatataaggctgtggcaaatcaatccacatacaacaacttcccagttttccttgatgtatgccatgcaaggaacttgtaattcataatgtaatagaattccatttgtttgccgtaaaaaaaaaaaaaaaaaa

SEQ ID NO: 6 - DNA, 1812 bp, Glycine max
ccaaactgaaccaaatcatcacacacacatactaagaagcaacaattcttctacaatggatctcctccttctggaaaagaccctcataggtctcttcctcgctgcggtggtcgccatcgccgtctccaccctccgcggccggaaattcaagctcccaccgggcccactccccgtcccaatcttcggcaactggctccaagtcggcgacgacctcaaccaccgcaacctcaccgatttggccaaaaaattcggtgacatcttcctcctccgcatggggcagcgcaacctcgtcgtggtttcttcccctgagctcgccaaagaggttctccacacgcagggcgtggagttcggctcccgcacccgcaacgtcgtcttcgacatcttcaccggaaagggccaagacatggtcttcaccgtctacggcgagcactggcgcaaaatgcgccgcatcatgaccgtccccttcttcaccaacaaggttgtgcaacaataccgccatggatgggaatcggaggctgccgccgtcgtcgaggacgtcaagaaaaaccccgacgccgccgtctccggcaccgtcatccgccgccgccttcagctcatgatgtacaacaacatgtaccgcataatgttcgaccggaggttcgagagcgaggaggatcccatcttccagaggctaagagccttgaacggagagaggagtcgcttggcgcagagctttgagtataactatggtgattttattcccatcttgagacccttcttgaagggttacttgaagatttgcaaggaggtgaaggagacgaggttgaagcttttcaaggattacttcgttgacgagaggaagaagcttggaagcaccaagagcaccaacaacaataatgaacttaaatgcgctattgaccacattttggatgcccagagaaaaggcgagatcaacgaagacaacgtcctctacattgttgaaaacatcaacgttgctgcaattgaaacaactctatggtcgattgagtggggcattgctgagcttgtgaaccacccagagatccagcaaaagttaagggatgagattgacagagttcttggagcagggcaccaagtgactgagccagacatccaaaagctcccatacctccaagcagtggtcaaggaaactcttcgtcttagaatggcaatccctctccttgtaccacacatgaacctccacgacgcaaagcttgggggctatgatatcccagctgagagcaagatcttggtgaatgcatggtggctggccaacaaccctgcacactggaagaagccagaggagttccggcctgagaggttcttcgaggaggagtcgcttgttgaagccaatggcaatgactttaggtaccttccctttggtgttggcagaagaagctgccctggaatcattcttgcattgccaattcttggcatcactttgggacgtttggtccaaaactttgagctcttgcctccccctggccagtcacagattgacactagtgagaaaggagggcaatttagcttgcacatactcaagcattccaccattgtggcaaagccaaggtcattttagactccaccacatgatcatgatcatatgatcaatccccttaattttttcttttttcttttcccttttctttactctgtattgtatcaatgcttgaaaatggggttgttccaaaaatgtcatatatatttggcctgctaatgggtattgtaaatctctgaacttgaagtgatgcacggttttgagatggttttctaataaacctacacttttgtctctaaaaaaaaaaaaaaaaaaaa

SEQ ID NO: 7 - DNA, 828 bp, Glycine max
gttaaatagaaaagaggagtttgagaatggcaacgatcagcgcggttcaggtggagttcctggagtttccagcggtggttacttcaccagcctccggcaagacctatttcctcggcggcgcaggggagagaggattgacgattgaggggaagttcataaagttcacaggcataggagtatacttggaggataaggcggtgccatcactcgccgctaagtggaagggtaaaacttcagaggagttagttcacaccctccacttctacagggatatcatttcagggccgtttgaaaagctaattagagggtcgaagattctgccattggctggcgctgaatactcaaagaaggtgatggaaaactgcgtggcacacatgaagtctgttgggacttacggtgatgctgaagccgcagccattgaaaagtttgctgaagccttcaagaatgtgaattttgcacctggtgcctctgttttctacagacaatcacctgatggaatcttggggcttagtttctctgaagatgcaacaataccagaaaaggaggctgcagtgatagagaacaaggctgtatcagcggcggtcttggagaccatgattggtgaacatgctgtttcccctgacttaaaacgcagtttggcttctcgattgcctgcggtattgagccacggcattatagtctgagaaatgagaaggatcaactttacctttttcaaatattcttgtttttctcctttctttcttgtcgcttgtcatgtatttctactgttttattaaataataaaattgagttctgttagagttggtgaaaaaaaaaaaaaaaaaaaaa

SEQ ID NO: 8 - DNA, 1394 bp, Glycine max
gttagaatggctgctgctattgaaatccccacaatagtgtttccaaactcctctgcccaacagaggatgccagtggttggaatgggatctgcccctgacttcacatgcaagaaagacacaaaggaggctatcattgaggccgtgaaacagggttacagacacttcgacactgctgctgcttatggctctgaacaggctctcggtgaagctctcaaggaagctatccatcttggcctcgtctcccgccaagacctctttgtcacttccaagctttgggtcaccgaaaatcatcctcatcttgtccttcctgctttgcgcaaatcacttaaaactcttcaactagagtacttggacctgtatctcatccactggcccctgagttctcagccagggaagttctcatttccaattgaagtagaagatctcttgccttttgacgtgaagggtgtgtgggaatccatggaagagtgccagaaacttggcctcaccaaagccattggagtcagcaacttctctgtcaagaagcttcagaatctgctctctgttgctaccatccgtcccgtggtcgatcaagtggagatgaaccttgcatggcaacagaagaagctaagagagttctgcaaagaaaatgggataatagtgactgcgttctctccactgaggaaaggtgcgagcaggggcccaaatgaagtgatggagaatgatgtgctgaaggagattgcagaggctcatgggaaatccatagcccaggtgagtctgagatggttgtacgaacaaggtgtgacatttgtgccaaagagctacgataaggagaggatgaaccagaatctgcacatctttgactgggcattgactgaacaagatcatcacaaaataagtcaaatcagccagagccgtttgatcagcggacccaccaaaccacaactcgctgatctctgggatgatcaaatataaactatttactactatgcagctcccactctatttttataatccatctttttacctcttgttcatttgtgccaaagagctacgataaggagaggatgaaccagaatctgcacatctttgactgggcattgactgaacaagatcatcccaaaataagtcaaatcagccagagccgtttgatcagcggaccccccaaaccacaactcgctgatctctgggatgatcaaatataaactatttactactatgcagctcccactctatttttataatccatctttttacctcttgtttcattttacgtttaaataattcatgccatgccacttcttattttagatttcacaatcaataaactaggcacgcgcggcacatgatatgaataaactatgttcaatttttttttcaaaaaaaaaaaaaaaaaaaaaaaaaaaaaaaaaaaa

SEQ ID NO: 9 - DNA, 1756 bp, Glycine max
gtaattaacctcactcaaactcgggatcacagaaaccaacaacagttcttgcactgaggtttcacgatgttgctggaacttgcacttggtttgtttgtgttagctttgtttctgcacttgcgtcccacaccaagtgcaaaatcaaaagcacttcgccacctcccaaaccctccaagcccaaagcctcgtcttcccttcattggccaccttcacctcttaaaagataaacttctccactatgcactcatcgatctctccaaaaagcatggccccttattctctctctccttcggctccatgccaaccgtcgttgcctccacccctgagttgttcaagctcttcctccaaacccacgaggcaacttccttcaacacaaggttccaaacctctgccataagacgcctcacttacgacaactctgtggccatggttccattcggaccttactggaagttcgtgaggaagctcatcatgaacgaccttctcaacgccaccaccgtcaacaagctcaggcctttgaggacccaacagatccgcaagttccttagggttatggcccaaagcgcagaggcccagaagccccttgacgtcaccgaggagcttctcaaatggaccaacagcaccatctccatgatgatgctcggcgaggctgaggagatcagagacatcgctcgcgaggttcttaagatcttcggcgaatacagcctcactgacttcatctggcctttgaagtatctcaaggttggaaagtatgagaagaggattgatgacatcttgaacaagttcgaccctgtcgttgaaagggtcatcaagaagcgccgtgagatcgtcagaaggagaaagaacggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaattaagataatcatcatatacaatagtagtgtcttgccatcgcagttgctttttatgtattcataatcatcatttcaataaggtgtgactggtacttaatcaagtaattaaggttacatacatgc

SEQ ID NO: 10 - DNA, 1465 bp, Glycine max
gcacgagggcacgaggaagcattgcattctgctatttaattccactacgtacacgcacattctcctcaaagacaacaatggcaccaacagccaagactctgacttacctggcccaggagaaaaccctagaatcgagcttcgttcgggacgaggaggagcgtcccaaggttgcctacaacgaattcagcgacgagatcccagtgatttctcttgccggaatcgacgaggtggatggacgcagaagagagatttgtgagaagatcgtggaggcttgcgagaattggggtatattccaggttgttgatcacggtgtggatcaacaactcgtggccgagatgacccgtctcgccaaagagttctttgctttgccaccggacgagaagcttcgttttgatatgtccggcgccaaaaagggtggattcattgtctccagccatctccaaggggaatcggtgcaggactggagagaaatagtgacatacttttcgtacccaaaaagagagagggactattcaaggtggccagacacgccagaagggtggagatcggtgactgaggaatacagcgacaaagtaatgggtctagcttgcaagctcatggaggtgttgtccgaagcaatggggttagagaaagagggtttaagcaaagcatgtgttgacatggaccagaaggtggtggttaattactaccccaaatgccctcaacctgacctcactcttggcctgaagcgccacacggatccgggcactatcaccttgctgcttcaggaccaagtgggtggacttcaagccaccagggacaatggcaaaacatggatcaccgttcagcctgtggaggctgccttcgtcgtcaatcttggagatcatgctcattatctgagcaatggaaggttcaagaatgctgatcaccaagcggtggtgaactcaaaccatagccgtttgtccatagccacttttcaaaacccagcaccaaatgcaactgtttaccctctgaagataagagaaggagagaagcctgtgatggaggaaccaatcacttttgctgaaatgtacaggaggaagatgagcaaggacattgagattgcaaggatgaagaagctggctaaggaaaagcatttgcaggaccttgagaatgaaaagcatttgcaagaacttgatcagaaggcaaaacttgaggccaagcctttgaaggagattcttgcttaattaataataattacatatgtatcatttgcatgcccccttggtgtttttagtattttttaagggccatgaattaataatagtccttacctttgtgcttttgtacgtcttatgatttatcctttgtggggatatcatgtgttgtgttcagttgcctatgtcttattagctagctggctcatctatgtataccttatatgtgcctctattataaatgaaaataagtggcactgtctttattaaaaaaaaaaaaaaaaaaaaaa

SEQ ID NO: 11 - DNA, 1279 bp, Glycine max
ataattcaactgttttggggtgtgattaaaagagaagctagctaaaaaaaatgggttcagcatccgaaagtgtttgcgttacaggagcttctggtttcatcgggtcatggcttgtcatgagactcatcgagcgtggctacaccgttcgagccaccgtacgcgacccagtaaacatgaagaaggtgaagcatttggtggaactaccaggcgcaaagagcaaactgtctctgtggaaggctgatcttgctgaagagggaagctttgatgaagccattaaaggctgcaccggagttttccacgtggccacccccatggactttgaatccaaagaccctgagaatgaagtgataaagcctacaataaatggggtactagacatcatgaaagcatgcttgaaggcaaaaactgtgcgaaggctaatattcacgtcctcagccggaaccctcaacgttattgagcgccaaaagcccgttttcgacgacacatgctggagtgacgttgagttttgccgtagagttaagatgactggttggatgtattttgtttctaaaacactggcggagaaagaagcatggaaatttgccaaagagcagggcctggacttcatcactatcattccacctcttgttgtcggtccctttctgatgccaaccatgccacctagcctaatcacggctctatcgccaatcacaggaaatgaggaccattactcgatcataaagcaaggtcaattcgtccacttagatgatctctgtcttgctcacatatttctgtttgaggaaccagaagtggaagggaggtacatatgcagtgcatgtgacgctaccattcatgacattgccaaattaattaaccaaaaataccctgagtacaaggtccccaccaagttcaagaatattccagatcaattggagcttgtgagattttcttccaagaagatcacagacttgggattcaaatttaaatacagcttagaggacatgtacactggagcaattgacacatgcagagacaaagggcttcttccgaaacctgcagaaaaagggctttttactaaacctggagaaactccagtgaatgccatgcataaataggcattcatatctttgtatctgtgtgatggctgtgcaacttgcttttcttattccgttgagtggcttttcttgattaacgtttctgttttatgaaaaattagaaatgtgagtggcttgtaaggccaggttatcttcaataagttaataaaaaccatcttctaaagtctaaaaaaaaaaaaaaaaaa

SEQ ID NO: 12 - DNA, 1234 bp, Glycine max
gcacgagattttatttttctttctttctttggaagataaagaatgggttctaagtccgaaaccgtttgcgttactggggcttctggttacatcggatcatggcttgtcatgagactcatcgagcgtggctataccgttcgagccaccgtactcgacccagctgatatgagggaggtgaagcatttgctggatctgccaggtgcagagagcaagctgtctctgtggaaggcagaacttacagaagagggaagctttgatgaagccattaaagggtgcacaggtgttttccacttggccacccccgttgactttaagtccaaagacccagagaatgaaatgataaagcctacaattcaaggagtactaaacatcatgaaagcatgcctgaaggcaaaaactgtccgaaggctagtattcacgtcctcagccggaactaccaacattactgagcaccaaaagcctatcattgacgaaacctgctggactgatgttgagttctgccggagattaaatatgactggttggatgtatttcgtttctaaaacacttgcggagaaagaagcttggaaatttgcgaaagagcacggcatggacttcatcgctatccttccagctcttgtcattggtccctttctactgccaacaatgccttctagcgtgatcagtgctctttcacctattaacggaattgaggcacattattcaatcataaagcaagctcaattcgtccacatagaagatatctgtcttgctcacatatttctgtttgaacagccaaaagcagaagggaggtatatatgcagtgcatgtgacgttactatccatgacattgtaaaattaattaacgaaaaatacccagagtacaaggttcccaccaagtttcagaacattccagatcaattggagcccgtgagattttcttccaagaaaatcacagacttgggattccaatttaaatacagcttagaggatatgtacactggagcaattgatacatgcatagagaaagggcttcttcctaaacctgcagaaattccagcgaatggcatcgagcataaataaatataggttttcatatctttgcctcggtgatggctatgaatgttgcttttcttgttcagtttctttaatgatgtttccgttttgtgaattcgtagtcaaaattgtaagtggtttgtaagaccaaattagttatctacaaattgtttaatattatcacaaaaaaaaaaaaaaa

SEQ ID NO: 13 - DNA, 1345 bp, Glycine max
gcacgagaatcaacacacacaaacacaacaacatatggaggtgctaagggtgcaaaccatagcttccaaatccaaagatgctgccatcccagccatgtttgttagggcagagacagagcaaccaggcatcacaaccgttcaaggggtgaaccttgaggtgccaattattgattttagtgacccagatgaagggaaagtggtgcatgagattttggaggcaagtagggactggggcatgttccaaattgtgaaccatgacatacctagtgatgttataagaaagttgcaaagtgttgggaaaatgttctttgagttgccacaagaggaaaaagagttgattgctaagcctgctgggtctgattctattgaagggtatggcacaaagcttcagaaagaggtgaatggcaagaaagggtgggtggatcatttgttccacattgtgtggcctccttcctccatcaactacagtttctggccccaaaaccccccttcttacagggaagttaatgaggaatattgcaagcacctaagaggagtggtagacaaattgttcaaaagtatgtcggtagggttggggcttgaagagaatgagctaaaggagggtgcaaatgaagatgacatgcattatcttttaaaaatcaattattacccaccttgtccatgtcctgatctggtcttgggtgtgccaccacacacagacatgtcctacctcacaattctggtgcctaacgaggtgcagggccttcaagcatgtagggatggccattggtacgatgttaagtatgtccccaatgccctcgttattcacattggcgaccaaatggagatactgagcaatggaaaatataaggcagtttttcacagaacaacagtgaacaaagatgagacaagaatgtcgtggcccgtgttcatagaacccaaaaaggaacaagaagttggtcctcacccaaagttggttaaccaagacaatccaccaaaatacaaaaccaagaaatacaaggattatgcttattgtaagctcaataagatccctcaatgaatgaagtgggcactacgagataattatagctcatggtttctctgttttgttatttaatttatggaatgaaagtgtgacttgtgggaagtagattaaataagatctgtgactaatgttggtttctctgttttgttatttaatttatggaatgaaagtgtgacttgtgggaagtagattaaataagatctgtgactaatgttggattaatcttatgcttttgaagtaaataaagatgacaagaaacagtttgtctgttctgttaaaaaaaaaaaaaaaaaaaaaaaaaaaaaaaaaaaaaaaaaaaaa

SEQ ID NO: 14 - DNA, 911 bp, Glycine max
agaccaagtcaagatcgttgcagcaataaaagaagctggaaacgtcaagagatttttcccatctgaatttgggctggatgtggaccgtcacgatgcggctgagcctgtaagagaagttttcgaggaaaaagcgaaaattcgaagagtaattgaagctgaaggaattccttacacttacctatgttgtcatgcctttactggttatttcttacgtaacctggcacagattgacatcactgttcctcctagggacaaggtgttcatacaaggagatggaaatgtgaaaggagcgtatattactgaggctgatgtgggaacttttaccatcgaagcagcaaatgaccctagagccttgaacaaagccgtgcacataagactcccaaacaattatttgtccttaaatgatatcatctctttgtgggagaaaaaaattgggaaaactcttgagaaaatttatgtttcagaagaagaagttcttaagcaaattaaggagacttctttcctaaataattatcttctggcactataccactcacagcagataaagggagatgcagtgtatgagattgaccctgccaaagaccttgaggcttctgaggcttatcctcacgtggaatacagcactgtttctgaatatttggatcagtttgtctgatttccgaaatcttaggaaccaaagcacttatatgattttatcactctgcaaaatgcttaataaaacaaatgcagttcttccttctgtttttctttcagaaaaggcctatgcaggcttttttgctgcactaccattgtctgtttcgtagtggttgcttgtgtttgtcctgttctcaagaaagtatccacatttaatcagtttaggtaagtgttctacctgaaaaaataataatataatcctgttttaatattgttac

SEQ ID NO: 15 - DNA, 21 bp, Artificial Sequence
  misc_feature (1)..(21): Oligonucleotide primer
ttgctggaacttgcacttggt

SEQ ID NO: 16 - DNA, 32 bp, Artificial Sequence
  misc_feature (1)..(32): Oligonucleotide primer
gtatatgatgggtaccttaattaagaaaggag

(19) United States
(12) Patent Application Publication
(10) Pub. No.: US 2003/0150012 A1
(43) Pub. Date: Aug. 7, 2003

(54) Method for altering the isoflavonoid profile in the plant parts of an isoflavonoid-producing plant

(76) Inventors: Joan T. Odell, Unionville, PA (US); Xiaodan Yu, St. Louis, MO (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/171,174

(22) Filed: Jun. 13, 2002

Related U.S. Application Data
(60) Provisional application No. 60/297,981, filed on Jun. 13, 2001.

(51) Int. Cl. A01H 5/00; A61K 35/78

(52) U.S. Cl. 800/278; 800/312; 800/313; 424/757

(57) ABSTRACT

A method for altering the ratio of total daidzein to total genistein in isoflavonoid-producing plants by using a C1 myb transcription factor and an R-type myc transcription factor is described. Also described are plant comprising these transcription factors in their genome as well as isoflavonoid-containing products made from seeds of these plants. Such products have an increased ratio of total daidzein to total genistein when compared to the total daidzein to total genistein ratio of a control.

This application claims priority to U.S. Provisional Application No. 60/297,981, filed Jun. 13, 2001 incorporated herein by reference in its entirety.

This invention pertains to methods of altering the ratios of individual isoflavonoids in isoflavonoid-producing plants by using a C1 myb transcription factor and an R-type myc transcription factor that regulate expression of genes in the phenylpropanoid pathway.

Isoflavonoids represent a class of secondary metabolites produced in legumes by a branch of the phenylpropanoid pathway and include such compounds as isoflavones, isoflavanones, rotenoids, pterocarpans, isoflavans, quinone derivatives, 3-aryl-4-hydroxycoumarins, 3-arylcoumarins, isoflav-3-enes, coumestans, alpha-methyldeoxybenzoins, 2-arylbenzofurans, isoflavanol, coumaronochromone and the like. In plants, these compounds are known to be involved in interactions with other organisms and to participate in the defense responses of legumes against phytopathogenic microorganisms (Dewick, P. M. (1993) in The Flavonoids, Advances in Research Since 1986, Harborne, J. B. Ed., pp. 117-238, Chapman and Hall, London). Isoflavonoid-derived compounds also are involved in symbiotic relationships between roots and rhizobial bacteria which eventually result in nodulation and nitrogen-fixation (Phillips, D. A. (1992) in Recent Advances in Phytochemistry. Vol. 26, pp 201-231, Stafford, H. A. and Ibrahim, R. K., Eds, Plenum Press, New York), and overall they have been shown to act as antibiotics, repellents, attractants, and signal compounds (Barz, W. and Welle, R. (1992) Phenolic Metabolism in Plants, pp 139-164, Ed by H. A. Stafford and R. K. Ibrahim, Plenum Press, New York).

Isoflavonoids have also been reported to have physiological activity in animal and human studies. For example, it has been reported that the isoflavones found in soybean seeds possess antihemolytic (Naim, M., et al. (1976) J. Agric. Food Chem. 24:1174-1177), antifungal (Naim, M., et al. (1974) J. Agr. Food Chem. 22:806-810), estrogenic (Price, K. R. and Fenwick, G. R. (1985) Food Addit. Contam. 2:73-106), tumor-suppressing (Messina, M. and Barnes, S. (1991) J. Natl. Cancer Inst. 83:541-546; Peterson, G., et al. (1991) Biochem. Biophys. Res. Commun. 179:661-667), hypolipidemic (Mathur, K., et al. (1964) J. Nutr. 84:201-204), and serum cholesterol-lowering (Sharma, R. D. (1979) Lipids 14:535-540) effects. These studies indicate that isoflavones in soybean protein products may produce many significant health benefits.

Free isoflavones rarely accumulate to high levels in soybeans. Instead they are usually conjugated to carbohydrates or organic acids. Soybean seeds contain three types of isoflavones in three different forms: the aglycones, daidzein, genistein and glycitein; the glucosides, daidzin, genistin and glycitin; and the malonylglucosides, 6″-O-malonyidaidzin, 6″-O-malonylgenistin and 6″-O-malonylglycitin. During processing acetylglucoside forms are produced: 6′-O-acetyidaidzin, 6′-O-acetyl genistin, and 6′-O-acetyl glycitin. The content of isoflavonoids in soybean seeds is quite variable and is affected by both genetics and environmental conditions such as growing location and temperature during seed fill (Tsukamoto, C., et al. (1995) J. Agric. Food Chem. 43:1184-1192; Wang, H. and Murphy, P. A. (1994) J. Agric. Food Chem. 42:1674-1677). In addition, isoflavonoid content in legumes can be stress-induced by pathogen attack, wounding, high UV light exposure and pollution (Dixon, R. A. and Paiva, N. L. (1995) Plant Cell 7:1085-1097). The genistein isoflavonoid forms make up the most abundant group in soybean seed and most food products, while daidzein and glycitein forms are present in lower levels (Murphy, P. A. (1999) J. Agric. Food Chem. 47:2697-2704).

The biosynthetic pathway for isoflavonoids in soybean and their relationship with several other classes of phenylpropanoids is presented in FIG. 1A and FIG. 1B.

Though the branch initiated by isoflavone synthase that leads to synthesis of isoflavonoids is mainly limited to the legumes, the phenylpropanoid pathway and other branches occur in other plant species. In maize, genes of the phenylpropanoid pathway and the lower anthocyanin branch are regulated by the transcription factor C1 in combination with an R-type factor. Together C1 and an R-type factor activate expression of a set of genes that leads to the synthesis and accumulation of anthocyanins in maize cells (Grotewold, E., et al. (1998) Plant Cell 10:721-740).

Maize C1 is a myb-type transcription factor that regulates expression of genes involved in anthocyanin production and accumulation in maize cells. However C1 cannot activate gene expression alone, and requires interaction with an R-type myc transcription factor for activation of target gene promoters. The R-type factors include, among others, alleles of R, alleles of the homologous B gene of maize, and alleles of the homologous Lc gene. These genes function similarly and make up the R/B gene family (Goff, S. A., et al. (1992) Genes Dev. 6:864-875). The various genes of the R/B gene family may be in turn each found as diverging alleles that fluctuate in expression pattern within the corn plant due to differences in their promoters. The members of this family encode proteins with very similar amino acid sequences and thus have comparable effects on the anthocyanin pathway structural genes. The specificity of the different promoters provides tissue specificity of anthocyanin biosynthesis (Radicella, J. P. et al. (1992) Genes Dev. 6:2152-2164; Walker, E. L. (1995) EMBO J. 14:2350-2363). The skilled artisan will recognize that the coding region of any functional gene of this large family could be used in conjunction with a promoter of choice to obtain R-gene function in the desired tissue or developmental stage. Examples of R/B family genes and alleles include, but are not limited to, Lc, R, R-S, R-P, Sn, B-Peru, and B-I. The coding regions of particular alleles of the Lc or B genes, especially the B-Peru allele, have been most commonly used in experiments in conjunction with C1.

Cell suspension lines of the maize inbred Black Mexican Sweet (BMS) that harbored an estradiol-inducible version of a fusion of C1 and R (CRC) were analyzed after the addition of estradiol. The cDNA fragments from the known flavonoid genes, except chalcone isomerase, were induced in the CRC-expressing line after hormone induction (Bruce et al. (2000) Plant Cell 12:65-80). Maize C1 and an R-type factor together can promote the synthesis of anthocyanins in Arabidopsis tissues that do not naturally express anthocyanins (Lloyd, A. M., et al. (1992) Science 258:1773-1775), and in petunia leaves (Quattrocchio, F., et al. (1993) Plant Cell 5:1497-1512).

WO 99/37794, published Jul. 29,1999, discloses the expression of maize C1 and the Lc allele of R in tomato fruit which led to increased levels of the flavonol kaempferol. Thus, it is known that C1 and an R-type factor can regulate expression of individual genes of the phenylpropanoid pathway in plants including Arabidopsis, petunia, tomato, and maize leading to production of anthocyanins or flavonols. These are all plants that do not produce isoflavones. Isoflavone production is almost exclusively limited to the legumes. An example of one of the few non-legume plants that does produce isoflavones is sugar beet.

C1 and B-Peru were transiently expressed in white clover and pea, which are legumes, (Majnik, et al. (1998) Aust. J. Plant Phys. 25:335-343) and anthocyanin levels assayed by visual inspection. Transient expression of C1 and B-Peru did result in production of anthocyanin in several tissues of white clover and pea. No assay was performed to determine any effect of C1 and B-Peru on isoflavonoid levels. Thus, any effects of C1 and an R-type myc on isoflavonoid levels in isoflavonoid-producing plants has not been taught.

WO 00/44909, published Aug. 3, 2000, discloses transformation of soybeans with maize C1 and R (as a CRC chimera) in conjunction with overexpression of the isoflavone synthase gene. Any effects of CRC alone on levels of isoflavonoids have not been reported. Thus, it is not known whether introduction of C1 and an R-type factor alone, without isoflavone synthase, could have any effect on the synthesis and accumulation of isoflavonoids in isoflavonoid-producing plants.

The physiological benefits associated with isoflavonoids in both plants and humans make the manipulation of their contents in crop plants highly desirable. For example, increasing levels of isoflavonoids in soybean seeds would increase the efficiency of extraction and lower the cost of isoflavone-related products sold today for use in either reduction of serum cholesterol or in estrogen replacement therapy.

In addition to altering the levels of total isoflavonoids, altering the ratios of individual isoflavonoid components is of interest. There is some indication that genistein and daidzein have individual effects in plant disease response and on human health. While daidzein is the precursor to the major phytoalexins of soybean, the glyceollins, genistein is involved in establishing the cell response to pathogen attack so that glyceollins are synthesized (Graham and Graham (2000) Mol. Plant Microbe Interact. 5:181-219). In human health, daidzein is effective in reducing levels of LDL-cholesterol and increasing the levels of HDL-cholesterol in human blood (U.S. Pat. No. 5,855,892). Daidzein is also effective for the treatment of hypertension and coronary atherosclerotic heart disease (Liu, Y., et al. (1990) Shenyang Yaoxueyuan Xuebao 7:123-125). Thus, raising the daidzein component in the total isoflavonoids could be valuable.

Therefore there is a need to enhance the level of isoflavonoids and to alter the ratios of isoflavonoid components in isoflavonoid-producing plants.

### SUMMARY OF THE INVENTION

This invention concerns a method of altering the isoflavonoid profile of an isoflavonoid-producing plant, said method comprising:

(a) transforming a plant with (i) a first recombinant expression construct comprising a promoter operably linked to an isolated nucleic acid fragment encoding a C1 myb transcription factor and a second recombinant expression construct comprising a promoter operably linked to an isolated nucleic acid fragment encoding an R myc-type transcription factor, (ii) a recombinant expression construct comprising a promoter operably linked to an isolated nucleic acid fragment encoding a C1 myb transcription factor and a promoter operably linked to an isolated nucleic acid fragment encoding an R myc-type transcription factor, or (iii) a recombinant expression construct comprising a promoter operably linked to an isolated nucleic acid fragment encoding all or part of a C1 myb transcription factor and all or part of an R myc-type transcription factor wherein said construct is capable of functioning as both a C1 myb transcription factor and an R myc-type transcription factor; and

(b) growing the transformed plant under conditions that are suitable for the expression of the recombinant expression construct or constructs; wherein expression of the construct or constructs alters the isoflavonoid profile of the transformed plant by increasing the total daidzein to total genistein ratio compared to the total daidzein to total genistein ratio of a control.

In a second embodiment, the recombinant expression construct described above comprises a promoter operably linked to an isolated nucleic acid fragment encoding a chimeric transcription factor comprising a maize R myc-type coding region situated between the C1 DNA binding domain and the C1 activation domain.

In a third embodiment, the isoflavonoid-producing plant is selected from the group consisting of soybean, clover, mung bean, lentil, hairy vetch, alfalfa, lupine, sugar beet, and snow pea. Also of interest are seed or plant parts of a plant transformed with a recombinant expression construct of the invention from which isoflavonoid-containing products can be obtained or extracted.

In a fourth embodiment, this invention concerns a food or beverage incorporating these isoflavonoid-containing products.

In a fifth embodiment, this invention concerns a method of producing an isoflavonoid-containing product which comprises: (a) cracking the seeds obtained from plants transformed with any of the recombinant expression constructs of the invention to remove the meats from the hulls; and (b) flaking the meats obtained in step (a) to obtain the desired flake thickness.

In a sixth embodiment, this invention concerns an isoflavonoid-producing plant comprising in its genome

(i) a first recombinant expression construct comprising a promoter operably linked to an isolated nucleic acid fragment encoding a C1 myb transcription factor and a second recombinant expression construct comprising a promoter operably linked to an isolated nucleic acid fragment encoding an R myc-type transcription factor,

(ii) a recombinant expression construct comprising a promoter operably linked to an isolated nucleic acid fragment encoding a C1 myb transcription factor and a promoter operably linked to an isolated nucleic acid fragment encoding an R myc-type transcription factor, or

(iii) a recombinant expression construct comprising a promoter operably linked to an isolated nucleic acid fragment encoding all or part of a C1 myb transcription factor and all or part of an R myc-type wherein said construct is capable of functioning as both a C1 myb transcription factor and an R myc-type transcription factor;

wherein said plant has an increased total daidzein to total genistein ratio when compared to the total daidzein to total genistein ratio of a control.

### BIOLOGICAL DEPOSIT

The following plasmid has been deposited under the terms of the Budapest Treaty with the American Type Culture Collection (ATCC), 10801 University Boulevard, Manassas, Va. 20110-2209, and bears the following designation, accession number and date of deposit.

| | Accession | |
|---|---|---|
| Plasmid | Number | Date of Deposit |
| pDP7951 | PTA371 | Jul. 29, 1999 |

### BRIEF DESCRIPTION OF THE DRAWINGS AND SEQUENCE LISTINGS

The invention can be more fully understood from the following detailed description and the accompanying Sequence Listing which form a part of this application.

FIG. 1A and FIG. 1B depict the soybean biosynthetic pathway for isoflavonoids and their relationship with several other classes of phenylpropanoids. FIG. 1A shows the pathway from phenylalanine to daidzein, genistein, and dihydroflavonol. FIG. 1B shows the pathway from daidzein, genistein, and dihydroflavonol to glyceollins, kievitone, anthocyanins, and flavonols.

FIG. 2 depicts the total daidzein to total genistein ratios observed for individual R1 seeds from plants obtained from four independent transformation events showing novel total daidzein to total genistein ratios and from control seeds. The source of the seed for each group (i.e. CRC transformation event number or control) is indicated above the bars. Control seeds are obtained either from a plant which was subject to bombardment and not found to contain the nucleic acid fragment of interest or from plants transformed with a recombinant DNA expression construct that does not alter the isoflavonoid profile of the transformed plant. Seeds 1, 2, 5,6, 7, 8, 9,10,11, 14,15, 16, 17, 21, 22, 23, 24, 25, 26, 27, 28,29, 31, 35, 36, 37, 39, 40, and 43 are from plants resulting from transformation experiments that, during PCR amplification, were negative for the CRC recombinant expression construct Seeds numbered 1 through 7 in FIG. 2 of the provisional application correspond to those numbered 3, 20, 8, 41, 13, 30, and 38 in this figure.

FIG. 3 depicts the total of isoflavone levels for individual R1 seeds obtained from plants from four independent transformation events showing novel total daidzein to total genistein ratios and from control seeds. The seeds in this figure are the same as those in FIG. 2. The source of the seeds for each group (i.e. control or event number) is indicated above the bars.

FIG. 4 depicts the total daidzein to total genistein ratios observed for single R2 seed from field-grown transgenic plants derived from CRC recombinant expression construct lines and from wild type segregants (indicated by an asterisk [*] on the figure). The seed with novel total daidzein to total genistein ratios also showed a brown stripe along the median. The source (CRC transformation event number) of the seed for each group is indicated above the bars.

FIG. 5 depicts the total daidzein to total genistein ratios observed for single R2 seed from a plant derived from the 1-1 transformation event and grown in a growth room. Seed without a brown stripe along the median are indicated with a pound sign (#) above the bars while unmarked bars represent seed with a brown stripe along the median.

FIG. 6 depicts the total isoflavone levels observed for single R2 seed from field-grown transgenic plants derived from CRC recombinant expression construct lines and from wild type segregants (indicated by an asterisk [*] on the figure). The source of the seed for each group (CRC transformation event number) is indicated above the bars.

FIG. 7 depicts the total isoflavone levels in single R2 seed obtained from a plant grown in a growth room and derived from the 1-1 transformation event. Seed without a brown stripe along the median are indicated with a pound sign (#) above the bars while the other, unmarked bars represent seed with a brown stripe. These represent the same individual seeds as in FIG. 5.

FIG. 8 depicts the total daidzein to total genistein ratios of bulk-analyzed R3 seed harvested from plants grown in a growth room. Each bulk seed sample is from a separate plant. The CRC recombinant expression construct line (i.e. CRC transformation event number) for each seed sample is indicated above the bars. Seed samples from wild type segregants derived from the CRC recombinant expression construct lines are indicated by an asterisk [*] above the bars.

FIG. 9 depicts the total isoflavone levels of bulk-analyzed R3 seed harvested from plants grown in a growth room. Each bulk seed sample is from a separate plant. The CRC recombinant expression construct line (i.e. CRC transformation event number) for each seed sample is indicated above the bars. Seed samples from wild type segregants derived from the CRC recombinant expression construct lines are indicated by an asterisk [*] on the figure. The seed are the same as those analyzed for FIG. 8.

FIG. 10 depicts the totals of individual isoflavones (daidzein, glycitein, and genistein) as well as the total isoflavones obtained from HPLC analyses of extracts prepared from individual R1 seeds obtained from plants transformed with the CRC recombinant DNA expression construct. Three to five seeds were analyzed from each plant. The control seeds are obtained from a transformant negative for the CRC recombinant DNA expression construct. Seeds obtained from plants positive for the CRC recombinant DNA expression construct are from individual transformation events 1-1, 1-2, 1-25, and 1-35. The source of the seeds for each group (transformation event followed by plant number) is indicated above the bars.

FIG. 11 depicts the ratios of total daidzein to the total isoflavones obtained for the same R1 seeds transformed with the CRC recombinant DNA expression construct and analyzed in FIG. 10.

FIG. 12 depicts the ratios of total genistein to the total isoflavones obtained for the same R1 seeds transformed with the CRC recombinant DNA expression construct and analyzed in FIG. 10.

FIG. 13 depicts the ratios of total daidzein to the total isoflavones obtained for individual R2 seeds from plants grown in the growth room and derived from three different transformation events (1-1, 1-2, and 1-25). The ratios obtained for six seeds from each plant are shown. The individual plants from which the seeds were harvested are identified with a number letter combination above the bars. The first two numbers designate the transformation event number, the third number designates the RO plant, and the letter designates the R1 plant from which the R2 seed were obtained. Seeds not having a brown stripe along the median are indicated with a pound sign (#) above the bar.

FIG. 14 depicts the ratios of total genistein to the sum of all isoflavones obtained for individual R2 seeds from plants grown in the growth room and derived from three different transformation events (1-1, 1-2, and 1-25). The ratios shown are for the same seed as shown in FIG. 13.

FIG. 15 depicts the ratios of total daidzein to the total isoflavones obtained for individual R2 seeds from plants grown in the field and derived from three different transformation events (1-1, 1-2, and 1-25). Each set of 2 seeds labeled with an asterisk (*) above the bar are tan seed from a segregant producing only tan seed, thereby identified as a wt segregant, of the transformation event of the adjacent plants. Three seeds all having a brown stripe were assayed from each of 2 CRC recombinant DNA expression construct-containing plants from each of the 3 transformation events. The individual CRC recombinant DNA expression construct-containing plants from which the seed were harvested are identified with three numbers. The first two numbers designate the transformation event number and the third number designates the RO plant from which the R2 seed were obtained.

FIG. 16 depicts the ratios of total genistein to the sum of all isoflavones obtained for individual R2 seeds from plants grown in the field and derived from three different transformation events (1-1,1-2, and 1-25). The ratios shown are for the same seeds as shown in FIG. 15.

FIG. 17 depicts the total of isoflavones obtained for individual R2 seeds from plants grown in the field and derived from three different transformation events (1-1, 1-2, and 1-25). The seeds are the same as those analyzed for FIG. 11.

FIG. 18 depicts an LC-MS2 mass chromatogram of m/z 504.6 to 505.6 obtained from extracts from a control wild type segregant seed without the CRC recombinant DNA expression construct.

FIG. 19 depicts an LC-MS2 mass chromatogram of m/z 504.6 to 505.6 obtained from extracts from brown striped R3 seed derived from the 1-1 transformation event. Additional peaks at 14.38, 15.46, 21.29, and 21.75 minutes are seen.

The following sequence descriptions and Sequences Listing attached hereto comply with the rules governing nucleotide and/or amino acid sequence disclosures in patent applications as set forth in 37 C.F.R. §1.821-1.825. The Sequence Listing contains the one letter code for nucleotide sequence characters and the three letter codes for amino acids as defined in conformity with the IUPAC-IUB standards described in Nucleic Acids Research 13:3021-3030 (1985) and in the Biochemical Journal 219 (No. 2):345-373 (1984) which are herein incorporated by reference.

The symbols and format used for nucleotide and amino acid sequence data comply with the rules set forth in 37 C.F.R. §1.822.

SEQ ID NO:1 is the nucleotide sequence of primer 1 used for detection of the CRC recombinant DNA fragment.

SEQ ID NO:2 is the nucleotide sequence of primer 2 used for detection of the CRC recombinant DNA fragment.

SEQ ID NO:3 is the nucleotide sequence of primer 3 used for the detection of genomic and chimeric isoflavone synthase genes.

SEQ ID NO:4 is the nucleotide sequence of primer 4 used for the detection of genomic and chimeric isoflavone synthase genes.

SEQ ID NO:5 is the nucleotide sequence of the cDNA insert in clone sdp3c.pkOO2.c22 encoding at least a portion of a soybean phenylalanine ammonia lyase.

SEQ ID NO:6 is the nucleotide sequence of the cDNA insert in clone src3c.pk014.e17 encoding at least a portion of a soybean cinnamic acid 4-hydroxylase.

SEQ ID NO:7 is the nucleotide sequence of the cDNA insert in clone ssm.pk0013.e3 encoding at least a portion of a soybean chalcone isomerase.

SEQ ID NO:8 is the nucleotide sequence of the cDNA insert in clone src3c.pk009.e4 encoding at least a portion of a soybean chalcone reductase.

SEQ ID NO:9 is the nucleotide sequence of the cDNA insert in clone pOY204 encoding at least a portion of a soybean isoflavone synthase.

SEQ ID NO:10 is the nucleotide sequence of the cDNA insert in clone sfl1.pk0040.gl 1 encoding at least a portion of a soybean flavanone 3-hydroxylase

SEQ ID NO:11 is the nucleotide sequence of the cDNA insert in clone sfl1.pk131.g5 encoding a portion of a soybean dihydroflavonol reductase.

SEQ ID NO:12 is the nucleotide sequence of the cDNA insert in clone src.pk0043.d11 encoding at least a portion of a soybean dihydroflavonol reductase.

SEQ ID NO:13 is the nucleotide sequence of the cDNA insert in clone ssl.pk0057.d12 encoding at least a portion of a soybean flavonol synthase.

SEQ ID NO:14 is the nucleotide sequence of the cDNA insert in clone srr1c.pk001.k4 encoding at least a portion of a soybean isoflavone reductase.

SEQ ID NO:15 is the nucleotide sequence of primer5 used for the preparation of an isoflavone synthase sequence by amplification from clone pOY204.

SEQ ID NO:16 is the nucleotide sequence of primer6 used for the preparation of an isoflavone synthase sequence by amplification from clone pOY204.

### DETAILED DESCRIPTION OF THE INVENTION

All patents, patent applications and publications cited are incorporated herein by reference in their entirety.

In the context of this disclosure, a number of terms shall be utilized.

The term “isoflavonoid(s)” refers to a large group of polyphenolic compounds, based on a common diphenylpropane skeleton, which occur naturally in plants. This term, as used herein, includes, but is not limited to, the three types of isoflavones in three different forms: the aglycones, daidzein, genistein and glycitein; the glucosides, daidzin, genistin and glycitin; and the malonylglucosides, 6″-O-malonyldaidzin, 6″-O-malonylgenistin and 6″-O-malonylglycitin, as well as, the acetylglucoside forms: 6′-O-acetyldaidzin, 6′-O-acetyl genistin, and 6′-O-acetyl glycitin that are formed during processing.

As used herein, “total genistein” refers to the total amount of this isoflavonoid regardless of the form. Thus, “total genistein” includes the aglycone form, the glucoside form, the malonylglucoside form, and other genistein forms. Likewise, “total daidzein” refers to the total amount of this isoflavonoid regardless of the form. Thus, “total daidzein” includes the aglycone form, the glucoside form, the malonylglucoside form, and other daidzein forms, and “total glycitein” includes the aglycone form, the glucoside form, the malonylglucoside form, and other glycitein forms.

The term “isoflavonoid-producing plant” refers to a plant in which isoflavonoids normally occur.

The term “control” refers to a plant or plant parts, such as seed, which is/are used as the basis for comparison. The control plant or plant parts, such as seed, described herein are plants or plant parts in which the isoflavone profile has not been altered. Examples of suitable controls include, but are not limited to, a wild-type plant or plant parts obtained from a wild type plant; a plant which was subject to bombardment and not found to contain the nucleic acid fragment or fragments of interest or a plant part, such as a seed or seeds, obtained from such a transformed plant; a control plant or plant part can be one derived from a transformed plant that contains the nucleic acid fragment or fragments of interest, but it does not now contain the nucleic acid fragment or fragments of interest due to segregation of the fragments(s) during sexual reproduction (this can be referred to as a wild-type segregant); or a control plant can be a plant transformed with a nucleic acid fragment that does not alter the isoflavone profile, e.g., a plant transformed to produce seeds with a high lysine phenotype but the isoflavone profile would not be altered. For example, if the plant of interest is a soybean plant then the preferred control would be seeds obtained from one of the plants described above. If the plant of interest is clover, then the preferred control would be leaves obtained from one of the plants described above. Those skilled in the art will appreciate that a particular control will depend upon the plant of interest.

The term “C1 myb transcription factor” refers to a protein encoded by a maize C1 gene and to any protein which is functionally equivalent to a C1 myb transcription factor.

The term “R myc-type transcription factor” refers to a protein with a basic helix-loop-helix domain encoded by a member of the R/B gene family and to any protein that is functionally equivalent to an R myc-type transcription factor.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of recombinant DNA fragments or chimeric genes to produce the desired phenotype in a transformed plant.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that substantially similar nucleic acid sequences encompassed by this invention are also defined by their ability to hybridize, under moderately stringent conditions (for example, 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

Sequence alignments and percent similarity calculations may be determined using a variety of comparison methods designed to detect homologous sequences including, but not limited to, the Megalign program of the LASARGENE bioinformatics computing suite (DNASTAR Inc., Madison, Wis.). Multiple alignment of the sequences are performed using the Clustal method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign gene” refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. An “allele” is one of several alternative forms of a gene occupying a given locus on a chromosome. When all the alleles present at a given locus on a chromosome are the same that plant is homozygous at that locus. If the alleles present at a given locus on a chromosome differ that plant is heterozygous at that locus.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro, J. K., and Goldberg, R. B. (1989) Biochemistry of Plants 15:1-82. It is further recognized that since in most cases the exact boundaries of regulatory sequences have not been completely defined, DNA fragments of some variation may have identical promoter activity.

The “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Mol. Biotech. 3:225-236).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The term “expression”, as used herein, refers to the production of a functional end-product, e.g., an mRNA or a protein (precursor or mature).

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms. The preferred method of cell transformation of rice, corn and other monocots is the use of particle-accelerated or “gene gun” transformation technology (Klein et al., (1987) Nature (London) 327:70-73; U.S. Pat. No. 4,945,050), or an Agrobacterium-mediated method using an appropriate Ti plasmid containing the transgene (Ishida Y. et al., 1996, Nature Biotech. 14:745-750).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

The term “recombinant” refers to an artificial combination of two otherwise separated segments of sequence, e.g., by chemical synthesis or by the manipulation of isolated segments of nucleic acids by genetic engineering techniques.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

A “recombinant DNA fragment” refers to an artificial combination of nucleic acid fragments that are not found together in nature, e.g. coding sequences and non-regulatory sequences. Thus, the difference between a “recombinant DNA fragment” and a “recombinant construct” as defined below turns on the presence or absence of regulatory sequences in the artificial combination of nucleic acid sequences. If a regulatory sequence is part of the combination then it is a “recombinant construct”. If there are no regulatory sequences in the combination, then it is a “recombinant DNA fragment”.

The terms “recombinant construct”, “expression construct”, “chimeric construct”, “construct” and “recombinant expression construct” are used interchangeably herein. A recombinant construct comprises an artificial combination of nucleic acid fragments, e.g., regulatory and coding sequences that are not found together in nature. For example, a chimeric construct may comprise at least one regulatory sequence and at least one coding sequence that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The present invention concerns a method of altering the isoflavonoid profile of an isoflavonoid-producing plant, said method comprising:

(a) transforming a plant with (i) a first recombinant expression construct comprising a promoter operably linked to an isolated nucleic acid fragment encoding a C1 myb transcription factor and a second recombinant expression construct comprising a promoter operably linked to an isolated nucleic acid fragment encoding an R myc-type transcription factor, (ii) a recombinant expression construct comprising a promoter operably linked to an isolated nucleic acid fragment encoding a C1 myb transcription factor and a promoter operably linked to an isolated nucleic acid fragment encoding an R myc-type transcription factor, or (iii) a recombinant expression construct comprising a promoter operably linked to an isolated nucleic acid fragment encoding all or part of a C1 myb transcription factor and all or part of an R myc-type transcription factor wherein said construct is capable of functioning as both a C1 myb transcription factor and an R myc-type transcription factor; and

(b) growing the transformed plant under conditions that are suitable for the expression of the recombinant expression construct or constructs; wherein expression of the construct or constructs alters the isoflavonoid profile of the transformed plant by increasing the total daidzein to total genistein ratio compared to the total daidzein to total genistein ratio of a control.

Also of interest are isoflavonoid-producing plants comprising in their genome

(i) a first recombinant expression construct comprising a promoter operably linked to an isolated nucleic acid fragment encoding a C1 myb transcription factor and a second recombinant expression construct comprising a promoter operably linked to an isolated nucleic acid fragment encoding an R myc-type transcription factor, (ii) a recombinant expression construct comprising a promoter operably linked to an isolated nucleic acid fragment encoding a C1 myb transcription factor and a promoter operably linked to an isolated nucleic acid fragment encoding an R myc-type transcription factor, or (iii) a recombinant expression construct comprising a promoter operably linked to an isolated nucleic acid fragment encoding all or part of a C1 myb transcription factor and all or part of an R myc-type wherein said construct is capable of functioning as both a C1 myb transcription factor and an R myc-type transcription factor; wherein said plant has an increased total daidzein to total genistein ratio when compared to the total daidzein to total genistein ratio of a control.

Examples of isoflavonoid-producing plants include, but are not limited to, soybean, clover, mung bean, lentil, hairy vetch, alfalfa, lupine, sugar beet, and snow pea. In a more preferred embodiment, the preferred isoflavonoid-producing plant would be soybean. Examples of other isoflavonoid-producing plants can be found in WO 93/23069, published Nov. 25, 1993, the disclosure of which is hereby incorporated by reference.

Transformation methods are well known to those skilled in the art and are described above.

The recombinant expression constructs which can be used to transform an isoflavonoid-producing plant fall into one of three categories:

(1) the constructs can be entirely separate, e.g., one construct may comprise a promoter operably linked to an isolated nucleic acid fragment encoding a C1 myb transcription factor and another separate construct may comprise a promoter operably linked to an isolated nucleic acid fragment encoding an R-myc type transcription factor;

(2) a single construct comprising a promoter operably linked to an isolated nucleic acid fragment encoding a C1 myb transcription factor and a promoter operably linked to an isolated nucleic acid fragment encoding an R-myc type transcription factor; or

(3) a single construct comprising a promoter operably linked to an isolated nucleic acid fragment encoding all or a part of a C1 myb transcription factor and an isolated nucleic acid fragment encoding all or a part of an R-myc type transcription factor such that a fusion protein combining the two encoded proteins is produced.

The transformed plant is then grown under conditions suitable for the expression of the recombinant expression construct or constructs. Expression of the recombinant expression construct or constructs alters the isoflavonoid profile of the transformed plant or plant part by increasing the total daidzein to total genistein ratio compared to the total daidzein to total genistein ratio of an untransformed plant or plant part. For example, in some cases it may be preferrable to examine expression of a recombinant expression construct by comparing seeds obtained from a transformed plant with seeds obtained from an untransformed plant to determine if there has been an increase in the total daidzein to total genistein ratio.

In a more preferred, embodiment, an isoflavonoid-producing plant can be transformed with a recombinant expression construct comprising a promoter operably linked to an isolated nucleic acid fragment encoding a chimeric transcription factor comprising the maize R coding region situated between the C1 DNA binding domain and the C1 activation domain.

The regeneration, development and cultivation of plants from single plant protoplast transformants or from various transformed explants is well known in the art (Weissbach and Weissbach, In.: Methods for Plant Molecular Biology, (Eds.), Academic Press, Inc., San Diego, Calif. (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens, and obtaining transgenic plants have been published for cotton (U.S. Pat. Nos. 5,004,863, 5,159,135, 5,518,908); soybean (U.S. Pat. Nos. 5,569,834, 5,416,011, McCabe et. al., BiolTechnology 6:923 (1988), Christou et al., Plant Physiol. 87:671-674 (1988)); Brassica (U.S. Pat. No. 5,463,174); peanut (Cheng et al., Plant Cell Rep. 15:653-657 (1996), McKently et al., Plant Cell Rep. 14:699-703 (1995)); papaya; and pea (Grant et al., Plant Cell Rep. 15:254-258, (1995)).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)).

Transient expression systems may be used to functionally dissect gene constructs (see generally, Maliga et al., Methods in Plant Molecular Biology, Cold Spring Harbor Press (1995)). It is understood that any of the nucleic acid molecules of the present invention can be introduced into a plant cell in a permanent or transient manner in combination with other genetic elements such as vectors, promoters, enhancers etc.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al., Molecular Cloning: A Laboratory Manual, Cold Spring Harbor Press (1989); Maliga et al., Methods in Plant Molecular Biology, Cold Spring Harbor Press (1995); Birren et al., Genome Analysis: Detecting Genes, 1, Cold Spring Harbor, N.Y. (1998); Birren et al., Genome Analysis: Analyzing DNA, 2, Cold Spring Harbor, N.Y. (1998); Plant Molecular Biology: A Laboratory Manual, eds. Clark, Springer, N.Y. (1997)).

Any promoter can be used in the method of the invention. Thus, the origin of the promoter chosen to drive expression of the coding sequence is not critical as along as it has sufficient transcriptional activity to accomplish the invention by expressing translatable mRNA for the desired protein genes in the desired host tissue. In a preferred embodiment, the promoter is a seed-specific promoter. Examples of a seed-specific promoter include, but are not limited to, the promoter for β-conglycinin (Chen et al. (1989) Dev. Genet. 10: 112-122), the napin and phaseolin promoters. A plethora of promoters are described in WO 00/18963, published on Apr. 6, 2000, the disclosure of which is hereby incorporated by reference.

Also within the scope of this invention are seeds or plant parts obtained from such transformed plants. Plant parts include differentiated and undifferentiated tissues, including but not limited to, roots, stems, shoots, leaves, pollen, seeds, tumor tissue, and various forms of cells and culture such as single cells, protoplasts, embryos, and callus tissue. The plant tissue may be in plant or in organ, tissue or cell culture.

In another aspect, this invention concerns an isoflavonoid-containing product high in total daidzein and low in total genistein obtained from the seeds or plant parts obtained from the transformed plants described herein. Examples of such an isoflavonoid-containing product include, but are not limited to, protein isolate, protein concentrate, meal, grits, full fat and defatted flours, textured proteins, textured flours, textured concentrates and textured isolates. In still another aspect, this invention concerns an isoflavonoid-containing product high in total daidzein and low in total genistein extracted from the seeds or plant parts obtained from the transformed plants described herein. An extracted product could then used in the production of pills, tablets, capsules or other similar dosage forms made to contain a high concentration of isoflavones.

Methods for obtaining such products are well known to those skilled in the art. For example, in the case of soybean, such products can be obtained in a variety of ways. Conditions typically used to prepare soy protein isolates have been described by [Cho, et al, (1981) U.S. Pat. No. 4,278,597; Goodnight, et al. (1978) U.S. Pat. No.4,072,670]. Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574] and Campbell et al. [(1985) in New Protein Foods, ed. by Altschul and Wilcke, Academic Press, Vol. 5, Chapter 10, Seed Storage Proteins, pp 302-338].

“Isoflavone-containing protein products” can be defined as those items produced from seed of a suitable plant which are used in feeds, foods and/or beverages. For example, “soy protein products” can include, but are not limited to, those items listed in Table 1. “Soy protein products”.

TABLE 1
Soy Protein Products Derived from Soybean Seeds^{a}

| | |
|---|---|
| Whole Soybean Products | Processed Soy Protein Products |
| Roasted Soybeans | Full Fat and Defatted Flours |
| Baked Soybeans | Soy Grits |
| Soy Sprouts | Soy Hypocotyls |
| Soy Milk | Soybean Meal |
| | Soy Milk |
| Specialty Soy Foods/Ingredients | Soy Protein Isolates |
| Soy Milk | Soy Protein Concentrates |
| Tofu | Textured Soy Proteins |
| Tempeh | Textured Flours and Concentrates |
| Miso | Textured Concentrates |
| Soy Sauce | Textured Isolates |
| Hydrolyzed Vegetable Protein | |
| Whipping Protein | |

^{a}See Soy Protein Products: Characteristics, Nutritional Aspects and Utilization (1987). Soy Protein Council

“Processing” refers to any physical and chemical methods used to obtain the products listed in Table 1 and includes, but is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale [(1990) Poult. Sci. 69:76-83]. “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No.10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338]. “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously [Atkinson (1970) U.S. Pat. No. 3,488,770, Horan (1985) In New Protein Foods, ed. by Altschul and Wilcke, Academic Press, Vol. 1A, Chapter 8, pp 367-414]. Moreover, conditions used during extrusion processing of complex foodstuff mixtures that include soy protein products have been described previously [Rokey (1983) Feed Manufacturing Technology III, 222-237; McCulloch, U.S. Pat. No. 4,454,804].

Also, within the scope of this invention are food and beverages which have incorporated therein an isoflavonoid-containing product of the invention.

The beverage can be a liquid or in a dry powdered form.

The foods to which the isoflavonoid-containing product of the invention can be incorporated/added include almost all foods/beverages. For example, there can be mentioned meats such as ground meats, emulsified meats, marinated meats, and meats injected with an isoflavonoid-containing product of the invention; nutritional supplements; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; and salad dressings; and dips and spreads such as mayonnaise; and chip dips; and food bars. The isoflavonoid-containing product can be added in an amount selected to deliver a desired dose to the consumer of the food and/or beverage. In still another aspect this invention concerns a method of producing an isoflavonoid-containing product which comprises: (a) cracking the seeds obtained from transformed plants of the invention to remove the meats from the hulls; and (b) flaking the meats obtained in step (a) to obtain the desired flake thickness.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Construction of Plasmids for Transformation of Glycine max

The effect on the isoflavonoid profile of soybean of a protein encoded by a recombinant DNA fragment containing maize nucleotide sequences encoding C1 and the Lc allele of R was tested. For this purpose, plasmid pOY203 was constructed for introduction of a CRC recombinant expression construct into soybean embryos. Plasmid pOY203 was briefly described in PCT publication WO 00/44090 (published Aug. 3, 2000) and contains a CRC recombinant DNA fragment under the control of the phaseolin promoter and termination signals in a vector containing expression systems which allow for selection for growth in the presence of hygromycin in both bacterial and plant systems.

Plasmid pOY203 was prepared through an intermediary plasmid pOY135. Plasmid pOY135 contains, flanked by Hind III restriction endonuclease sites, the CRC recombinant DNA fragment inserted between the phaseolin promoter and polyadenylation signal sequences. The CRC recombinant DNA fragment contains, between Sma I sites and in the 5′ to 3′ orientation, maize nucleotide sequences encoding

(a) the C1 myb domain to amino acid 125;

(b) the entire coding region of the Lc allele of R (amino acids 1 through 160); and

(c) the C1 transcription activation domain (from amino acid 126 to the C-terminus of C1).

The CRC recombinant DNA fragment was isolated from plasmid pDP7951 (described in PCT Publication WO 00/44090, published Aug. 3, 2000, and bearing ATCC deposit No. PTA371) and inserted into vector pCW108N. Vector pCW108N is derived from the commercially-available vector pUC18 (Gibco-BRL) and contains between Hind III sites:

(a) a DNA fragment of the phaseolin gene promoter extending from −410 to +77 relative to the transcription start site (Slightom et al. (1991) Plant Mol. Biol. Man. B16:1); and

(b) a 1175 bp DNA fragment including the polyadenylation signal sequence region of the same phaseolin gene (see sequence descriptions in Doyle et al. (1986) J. Biol. Chem. 261:9228-9238 and Slightom et al. (1983) Proc. Natl. Acad. Sci. USA 80:1897-1901).

Plasmid pCW108N was digested with Asp 718, which cuts between the phaseolin promoter and polyadenylation signal sequence, and the protruding ends filled-in by incubation with T4 DNA polymerase in the presence of dATP, dCTP, dGTP, and dTTP. The DNA fragment containing the CRC recombinant DNA fragment was isolated from pDP7951 by digestion with Sma I, purified by agarose gel electrophoresis, and inserted into the blunt-ended pCW108N to create pOY135.

To create pOY203, a cassette containing the phaseolin promoter/CRC recombinant DNA fragment/phaseolin polyadenylation signal sequence (herein referred to as CRC recombinant expression construct) was liberated from pOY135 by digestion with Hind III and introduced into Hind III-digested pZBL102. Plasmid pZBL102 contains expression systems which allow for selection for growth in the presence of hygromycin to be used as a means of identifying cells that contain plasmid DNA sequences in both bacterial and plant systems and is described in PCT Publication WO 00/44090.

Even though it is not necessary for the practice of the invention, in the original experiment, plasmid pOY203 was co-bombarded into soybean embryos with plasmid pWSJ001 also described in PCT publication WO 00/44090. Plasmid pWSJ001 contains the isoflavone synthase coding region under the control of the alpha' beta-conglycinin promoter and phaseolin polyadenylation signal sequence in a vector containing expression systems which allow for selection for growth in the presence of hygromycin in both bacterial and plant systems. The isoflavone synthase coding region (found in NCBI General Identifier No. 6979520) was obtained by PCR amplification of a clone (sgs1c.pk006.o20) obtained from a soybean cDNA library prepared from seeds germinated for 4 hours. Amplification was performed using Pfu polymerase (Stratagene) in a standard PCR reaction in a GeneAmp PCR System with primer5 (shown in SEQ ID NO:15) and primer6 (shown in SEQ ID NO:16).

[SEQ ID NO:15]

5′-TTGCTGGAACTTGCACTTGGT-3′

[SEQ ID NO:16]

5′-GTATATGATGGGTACCTTAATTAAGAAAGGAG-3′

The isoflavone synthase coding region was first inserted between the alpha' beta-conglycinin promoter and phaseolin polyadenylation signal sequence of vector pCW109. Vector pCW109 contains a 550 bp fragment of the alpha' beta-conglycinin promoter (Slightom et al. (1991) Plant Mol Biol. Man. B16:1) and the same phaseolin polyadenylation signal sequence described above for pCW108N. The Nco I site located between the promoter and polyadenylation signal sequence fragments in plasmid pCW109 was eliminated by digestion with Nco I followed by fill-in with T4 DNA polymerase in the presence of dATP; dCTP, dGTP and dTTP. The resulting DNA was digested with Kpn I, which cuts 3′ of the filled-in Nco I site, and the isoflavone synthase fragment introduced. The cassette containing the IFS chimeric gene (alpha' beta-conglycinin promoter/isoflavone synthase/phaseolin 3′ polyadenylation sequence) was liberated from this plasmid by digestion with Hind Ill and introduced into Hind III-digested pZBL102 to form pWSJ001.

### Example 2

### Transformation of Somatic Soybean Embryo Cultures and Regeneration of Soybean Plants

The ability to alter the isoflavone levels in transgenic soybean plants expressing the CRC recombinant expression construct was tested by transforming soybean somatic embryo cultures with plasmids pOY203 and pWSJ001, screening for transformants expressing only the CRC recombinant expression construct, allowing plants to regenerate, and measuring the levels of isoflavones produced. The present invention does not require the presence of plasmid pWSJ001. Screening for the presence of the transgenes was performed by PCR amplification, and plants containing the isoflavone synthase recombinant-expression construct were excluded from this work.

Soybean embryogenic suspension cultures were transformed with pOY203 in conjunction with pWSJ001 by the method of particle gun bombardment, and transformants carrying the CRC recombinant expression construct in pOY203, and not the IFS recombinant expression construct in pWSJ001, were identified.

The following stock solutions and media were used for transformation and regeneration of soybean plants:

### Stock Solutions (per Liter)

MS Sulfate 100×stock: 37.0 g MgSO₄.7H₂O, 1.69 g MnSO₄.H₂O, 0.86 g ZnSO₄.7H₂, 0.0025 g CuSO₄.5H₂O.

MS Halides 100×stock: 44.0 g CaCl₂.2H₂O, 0.083 g KI, 0.00125 g CoCl₂.6H₂O, 17.0 g KH₂PO₄, 0.62 g H₃BO3, 0.025 g Na₂MoO₄.2H₂O, 3.724 g Na₂EDTA, 2.784 g FeSO₄.7H₂O.

B5 Vitamin stock: 100.0 g myo-inositol, 1.0 g nicotinic acid, 1.0 g pyridoxine HCl, 10.0 g thiamine.

2,4-D stock: 10 mg/mL

### Media (per Liter)

SB55: 10 mL of each MS stock, 1 mL of B5 Vitamin stock, 0.8 g NH₄NO₃, 3.033 g KNO₃, 1 mL 2,4-D stock, 0.667 g asparagine, pH 5.7.

SB103: 1 pk. Murashige & Skoog salt mixture (Gibco BRL), 60 g maltose, 2 g gelrite, pH 5.7.

SB71-1: B5 salts,1 mL B5 vitamin stock, 30 g sucrose, 750 mg MgCl2, 2 g gelrite, pH 5.7.

Soybean (of the Jack variety) embryogenic suspension cultures were maintained in 35 mL SB55 liquid media on a rotary shaker (150 rpm) at 28° C. with a mix of fluorescent and incandescent lights providing a 16 hour day, 8 hour night cycle. Cultures were subcultured every 2 to 3 weeks by inoculating approximately 35 mg of tissue into 35 mL of fresh liquid media.

Soybean embryonic suspension cultures were transformed by the method of particle gun bombardment (see Klein et al. (1987) Nature 327:70-73) using a DuPont Biolistic PDS1000/He instrument. Embryos were co-bombarded with plasmid pOY203 (containing the CRC recombinant expression construct) and plasmid pWSJ001 (containing the IFS recombinant expression construct). Transformants containing the CRC recombinant expression construct alone were identified by PCR and are described herein. Transformants containing the IFS recombinant expression construct were used for other purposes and do not form part of the present invention.

For bombardment, 5 μL of a 1:2 mixture of pOY203 (0.5 μg/μL) and pWSJ001 (1 μg/μL) plasmid DNA, 50 μL CaCl₂ (2.5 M), and 20 μL spermidine (0.1 M) were added to 50 μL of a 60 mg/mL 0.6 μm gold particle suspension. The particle preparation was agitated for 3 minutes, spun in a microfuge for 10 seconds and the supernatant removed. The DNA-coated gold particles were then washed once with 400 μL of 100% ethanol, resuspended in 40 μL of anhydrous ethanol, and sonicated three times for 1 second each. Five μL of the DNA-coated gold particles was then loaded on each macro carrier disk.

Approximately 300 to 400 mg of two-week-old suspension culture was placed in an empty 60 mm×15 mm petri dish and the residual liquid removed from the tissue using a pipette. The tissue was placed about 3.5 inches away from the retaining screen and bombarded twice. Membrane rupture pressure was set at 1 100 psi and the chamber was evacuated to −28 inches of Hg. Two plates were bombarded for each experiment and, following bombardment, the tissue was divided in half, placed back into liquid media, and cultured as described above.

Eleven days after bombardment, the liquid media was exchanged with fresh SB55 media containing 50 mg/mL hygromycin. The selective media was refreshed weekly. Seven weeks post bombardment, green, transformed tissue was observed growing from untransformed, necrotic embryogenic clusters. Isolated green tissue was removed and inoculated into individual flasks to generate new, clonally propagated, transformed embryogenic suspension cultures. Thus, each new line was treated as an independent transformation event. Soybean suspension cultures can be maintained as suspensions of embryos clustered in an immature developmental stage through subculture or can be regenerated into whole plants by maturation and germination of individual somatic embryos.

Transformed embryogenic clusters were removed from liquid culture and placed on SB103 solid agar media containing no hormones or antibiotics. Embryos were cultured for eight weeks at 26° C. with mixed fluorescent and incandescent lights on a 16 hour day, 8 hour night schedule. During this period, individual embryos were removed from the clusters and analyzed at various stages of embryo development. Selected lines were assayed by PCR amplification for the presence of the CRC recombinant expression construct and/or the IFS recombinant expression construct.

| | |
|---|---|
| 5′-AGGCGGAAGAACTGCTGCAACG-3′ | [SEQ ID NO:1] |
| 5′-AGGTCCATTTCGTCGCAGAGGC-3′ | [SEQ ID NO:2] |
| 5′-ATGTTTGGCAAGTAGGAAGGGACC-3′ | [SEQ ID NO:3] |
| 5′-GCATTCCATAAGCCGTCACGATTC-3′ | [SEQ ID NO:4] |

The presence of the CRC recombinant expression construct was determined using primer1 and primer2 (shown in SEQ ID NO:1 and SEQ ID NO:2, respectively) which produce a fragment that is not present in wild type soybean embryos. The presence of the IFS recombinant expression construct was determined using primer3 and primer4 (shown in SEQ ID NO:3 and SEQ ID NO:4, respectively). Separation, on an agarose gel, of the amplification products obtained with this pair of primers yielded a 1062 bp fragment indicative of the endogenous IFS gene (i.e., containing introns) in all samples and an 845 bp fragment in the embryos also containing the IFS recombinant expression construct. Embryos containing the CRC recombinant expression construct and not the IFS recombinant expression construct were selected for further study.

Somatic embryos became suitable for germination after eight weeks and were then removed from the maturation medium and dried in empty petri dishes for 1 to 5 days. The dried embryos were then planted in SB71-1 medium where they were allowed to germinate under the same lighting and germination conditions described above. Germinated embryos were transferred to sterile soil and grown to maturity. Seeds were harvested.

### Example 3

### Analysis of Isoflavones in R1 Seed of Transformants Containing the CRC Recombinant Expression Construct

Isoflavone levels were analyzed in seed from soybean primary transformants (R1 seed) containing the CRC recombinant expression construct and not the IFS recombinant expression construct. Extracts were prepared and analyzed by HPLC as follows. Each seed was weighed and placed in a 2 mL screw cap tube containing a ¼″ cylindrical bead and 20 mg flavone (as internal standard). The seed was then crushed using a bead beater at 4200 rpm for 30 second intervals until reduced to a fine powder. The sample was homogenized into solution by the addition of 800 μL of 80% aqueous methanol and further bead beating. Each sample was left in a shaking water bath at 60° C. for 4 hours and then centrifuged at 12000 rpm for 10 minutes. A 100 μL aliquot of the supernatant was removed and added to 100 μL deionized water, vortexed, centrifuged, and analyzed by HPLC. An HP 1100 instrument equipped with a diode array detector and a Phenomenex, Luna 3 C18(2), 4.6 mm×150 mm column was used for HPLC analysis. The column temperature was 22° C., the solvent flow rate was 1 mL/min, and the detection was performed at 260 nm. The solvent elution consisted of a gradient from 5% methanol/ 95% 0.1% trifluoroacetic acid (TFA) in water to 100% methanol over 16 minutes followed by a 3 minute post-run wash. This resulted in chromatograms depicting daidzein, glycitein, genistein, and their conjugate derivatives. Standard curves were constructed with each analysis and individual compounds were measured. All of the conjugates were converted to aglycone equivalent values using standard conversion factors. In addition to total concentrations for each aglycone, the total isoflavone content was also calculated.

Generally, five individual seeds from each of one to three plants from each transformation event were analyzed. Seeds from primary transformant plants from a total of 13 transformation events, each carrying only the CRC recombinant expression construct, were analyzed as well as seed from primary transformant plants not carrying a transgene. A subset of the CRC events showed an altered isoflavonoid composition as compared to the controls. Observing a phenotype in a portion of transgenic plants is explained by the usual variation of expression of a transgene that occurs in independent transformation events.

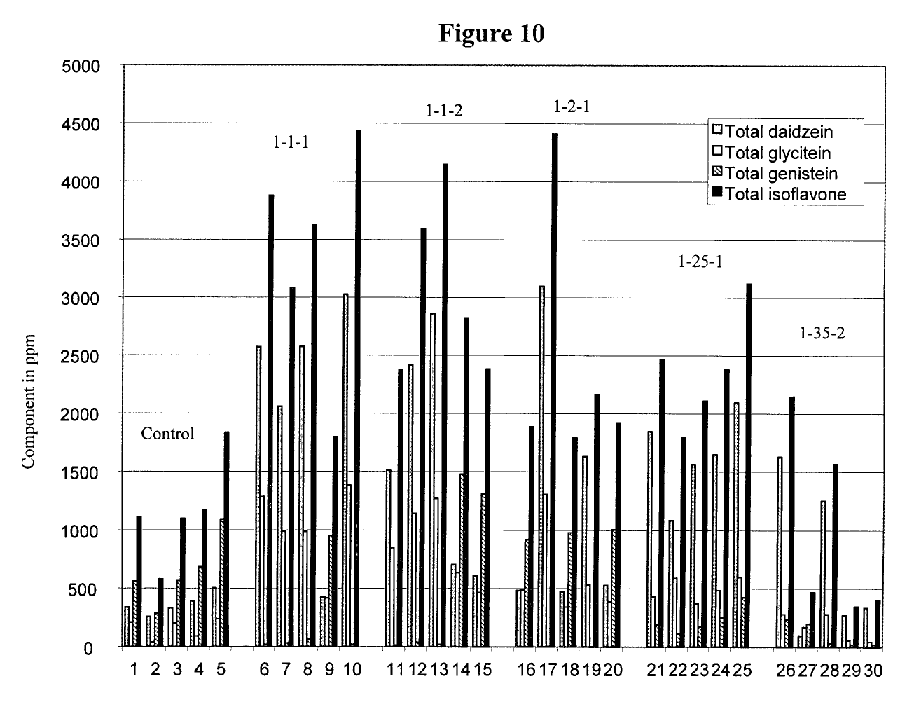

The isoflavone component profile for seeds from a control plant is shown in FIG. 10, seeds #1-5. This control plant came from a transformation experiment but it was PCR negative for the CRC recombinant expression construct. In this typical control profile, genistein is the most abundant of the isoflavones. Daidzein is generally the next highest level component with glycitein lowest, although in some seeds the daidzein and glycitein levels can be similar. This example also shows a substantial amount of variation in the levels of the individual isoflavones, as well as the sum of all isoflavone levels, among individual seeds from the same plant. An obvious change in the isoflavone component profile could be seen in seeds obtained from plants representing four independent transformation events (FIG. 10). The R1 seeds from the hemizygous primary transformants would be expected to be segregating for the transgene. Among the seeds analyzed from the 1-1, 1-2, and 1-35 event plants there are seeds with an altered profile as well as seeds with the control profile described above. All of the seeds from the 1-25 event had an altered profile indicating that all five seeds contain the transgene. This could be due to the presence of multiple segregating loci or due to the selection by chance of five single seeds, each containing a single locus.

The glycitein levels were the least affected in these seeds with altered isoflavone components. However in some seeds, particularly of the 1-1 event, the glycitein levels were increased about two-fold above the level in wild type segregant seeds of the same transformant (FIG. 10, seeds # 6, 10, 13). The pathway for glycitein synthesis is not defined, but may be a part of the daidzein branch due to more similarity in glycitein structure with daidzein than with genistein. An enzyme encoded by the CYP71 D9 P450 that may be involved in glycitein synthesis was recently characterized (Latunde-Dada et al. (2001) J. Biol. Chem. 276: 1688-1695). If daidzein and glycitein are closely related, the CRC transgene has the effect of activating the daidzein/glycitein branch of isoflavone synthesis. In some individual seeds from CRC transformants having high daidzein levels the total isoflavone levels were increased. Out of the 16 seeds with altered isoflavone profiles (shown in FIG. 10), 14 seeds had higher total isoflavone levels than the seeds from the control plant. It may be concluded that the total isoflavone level in individual seeds is quite variable, but CRC can, in some cases, raise the level further. The inconsistency of this effect suggests that there must be other factors that contribute to establishing the final total isoflavone levels.

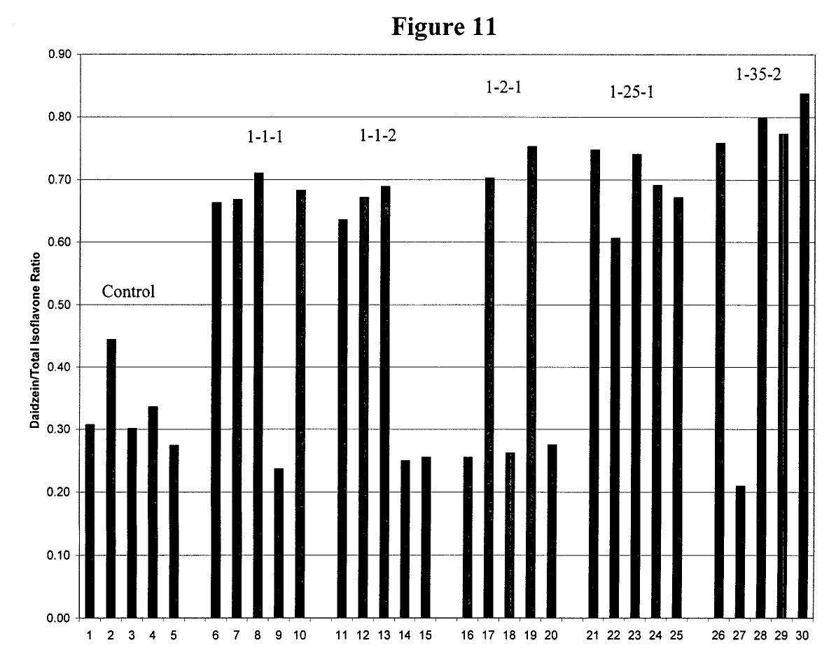

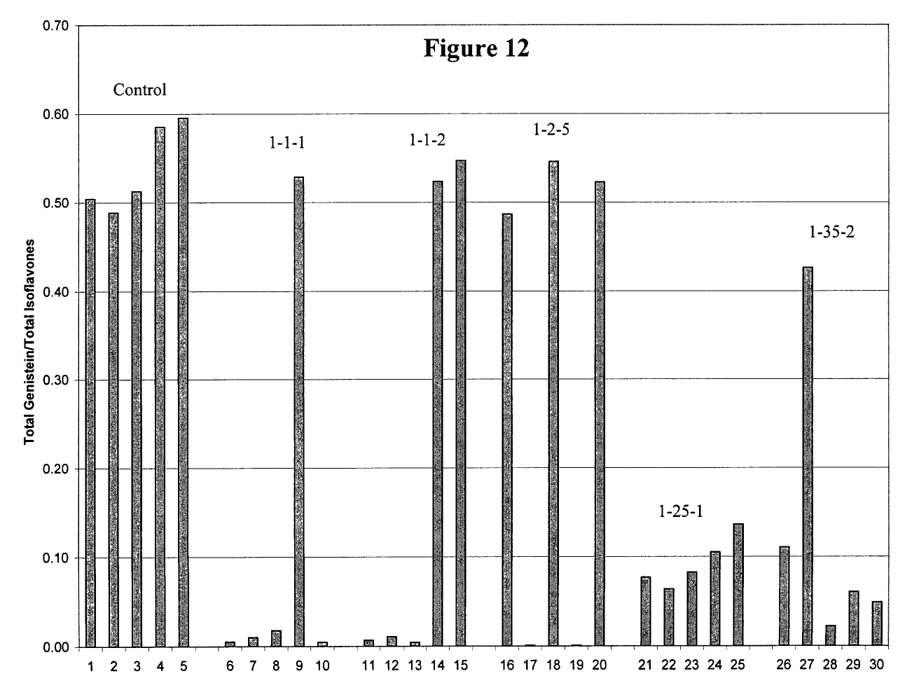

The altered isoflavone profile of seeds in these four events is distinguished by greatly increased levels of total daidzein, the highest level being raised about four-fold when compared to the daidzein levels in control and wild type segregating seeds (seeds #10 and 17). The same individual seeds with high levels of daidzein also had greatly decreased levels of genistein, in some instances decreased to almost undetectable levels (seeds # 6,11, 13, for example). These changes result in the daidzein component contributing 60% to 80% of the total isoflavones in the altered phenotype seeds, while daidzein is generally 20% to 35% of the total in control and wild type segregating seeds (FIG. 11). In the altered phenotype seeds the genistein component ranges from a low of almost 0% up to 14%, while in control and wild type segregating seeds the range is between 43% and 60% of the total isoflavones (FIG. 12). The reduction in genistein level varied between the different transformation events, with event 1-2 having the greatest genistein reduction, almost to zero. In event 1-25 genistein was only reduced to between 6% and 14% of total isoflavones, this is still much below control levels.

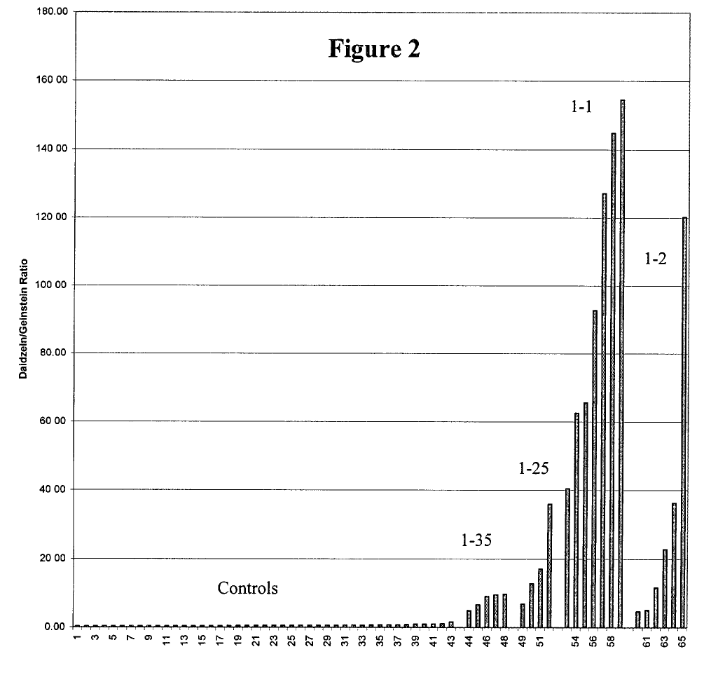

FIG. 2 shows the total daidzein to total genistein ratios for individual seeds obtained from plants from the 1-1,1-2,1-25, and 1-35 transformation events having an increased total daidzein to total genistein ratio as well as from control seeds. The control seeds are obtained either from plants resulting from transformation experiments that, during PCR amplification, were negative for the CRC recombinant expression construct, or from plants transformed with a recombinant DNA expression construct that does not alter the isoflavonoid profile. The ratios for two seeds obtained from the 1-2 event are not shown because their ratios are too high (784.0 and 801.0) to plot on the same chart. While the total daidzein to total genistein ratios for control seeds ranged between 0.3 and 1.6, the ratios for the seeds from the four transformation events with the novel high total daidzein phenotype ranged between 4.7 and 801.0. The exact ratio of total daidzein to total genistein was variable between individual seeds, even within a single transformation event. However, it is clear that expression of the CRC recombinant expression construct in soybean seeds altered the total daidzein to total genistein ratio from being less than 2, to being over 4.5.

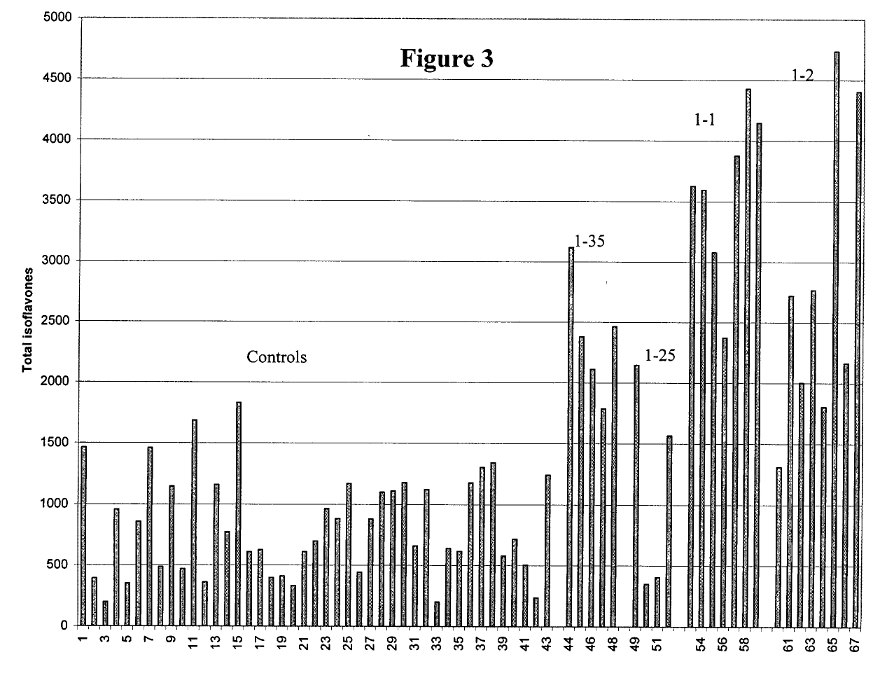

Most of the seeds from the four transformation events having increased total daidzein to total genistein ratios also had increased levels of total isoflavones. FIG. 3 depicts a graph showing the total isoflavone levels for the same seeds as the total daidzein to total genistein ratios are shown for in FIG. 2. Of 24 seeds analyzed that showed high total daidzein to total genistein ratios, 18 had total isoflavone levels higher than the highest total isoflavone values for the controls. The control seeds had total isoflavonee levels ranging from 199 to 1833 μg/g seed weight. Eighteen of the seeds showing increased total daidzein to total genistein ratios had total isoflavone levels between 2003 and 4737, while the remaining six seeds showing increased total daidzein to total genistein ratios had total isoflavone levels between 348 and 1808. Thus, expression of the CRC recombinant expression construct in soybean seeds produced higher levels of total isoflavones in a majority of the seeds having high total daidzein to total genistein ratios.

### Example 4

### Analysis of Isoflavones in R2 Seed of Transformants Containing the CRC Recombinant Expression Construct

R1 seeds from the 1-1, 1-2,1-25 and 1-35 events described above were planted in the field at the Stine location in Newark, DE and R1 seeds from the 1-1 event were also planted in pots and grown in a growth room. Seeds were harvested (R2 seed) and analyzed for isoflavone levels. Single seed extracts were prepared and analyzed as described in Example 3 with the following modifications. No internal standard was added. Samples were extracted in 80% methanol for 1 hour at 27° C. After centrifugation, 500 μL of supernatant was transferred to a fresh 2 mL tube. An additional 500 μL of 80% methanol was added to the ground seed left in the tube, the mix was resuspended for 30 seconds using a Spex 2000 Geno-grinder at 1620 strokes/min, and the centrifugation repeated. Another 500 μL of supernatant was combined with the 500 μL in the fresh tube, the sample vortexed, centrifuged again, and 300 μL added to 300 μL of deionized H₂O and vortexed. The sample was assayed by HPLC under the conditions of Example 3 except that the column temperature was 25° C., and the detection was at 262 nm. The data was calculated as described in Example 3.

It was noted that individual seeds with the high total daidzein to total genistein ratio also had a brown stripe along the median of the seed. These seeds had a dark brown stripe around the median on the side opposite to the hilum, parallel to the cotyledon axis, as opposed to the overall light tan of seeds having a control phenotype. Some of the brown striped seeds were smaller than control seeds and some were slightly wrinkled. Cutting the seeds showed that the brown pigmentation was only on the external coat and did not extend into the cotyledons.

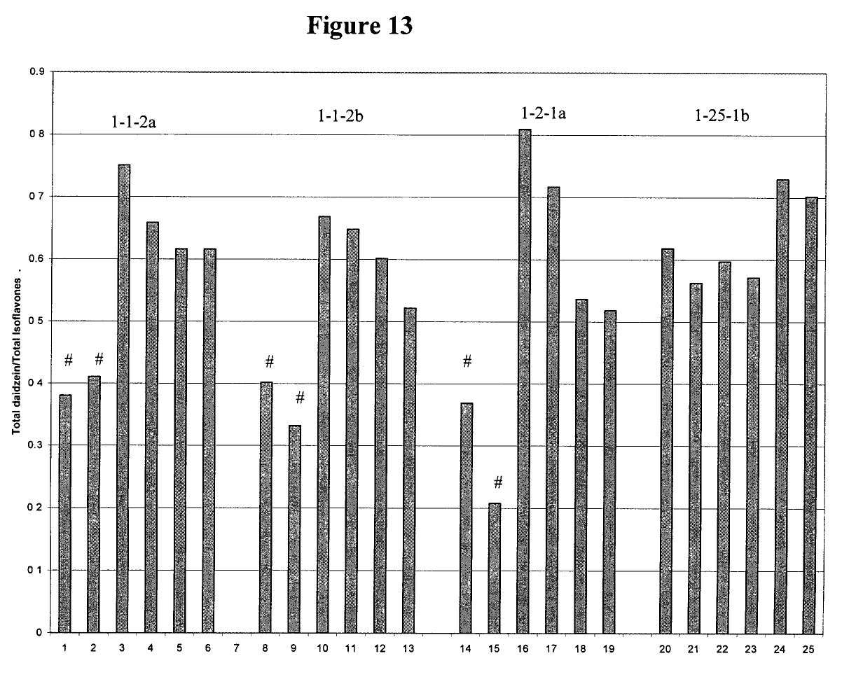

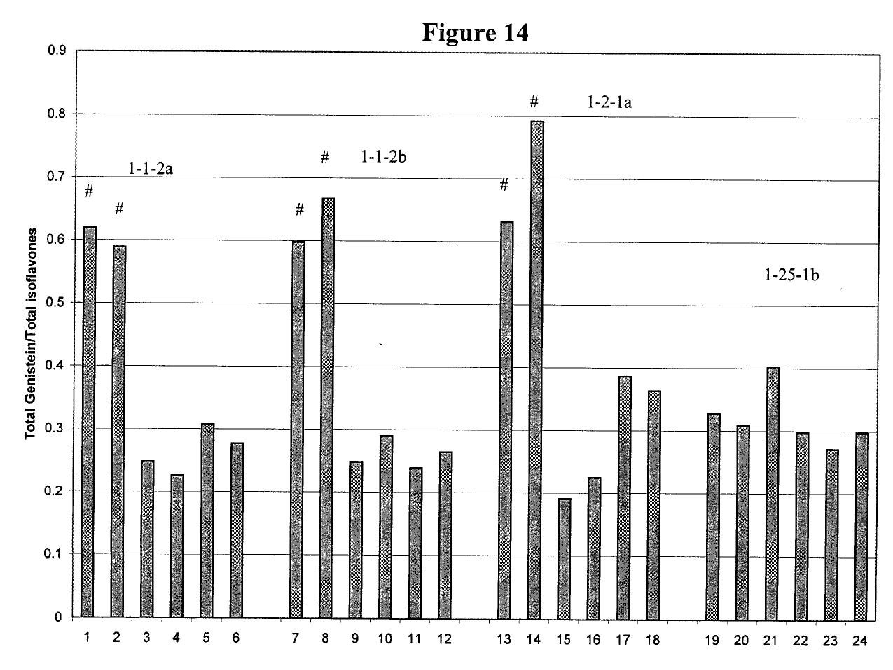

To further investigate a possible correlation between the visual phenotype and isoflavone profile, plants were grown in the growth chamber from either tan or brown striped RI seeds from the 1-1, 1-2, and 1-25 events and the harvested R2 seeds observed. All plants grown from tan seeds produced only tan seeds. The 1-25 plant grown from a brown stripe seed produced 17 brown striped seeds, consistent with there being multiple loci in this line. The 1-1 and 1-2 plants grown from brown striped seeds all produced segregating brown striped and tan seeds. For the 1-2 line the segregation was 3:1 brown striped to tan, indicating a dominant trait. For the 1-1 line, the segregation ratio was 2:1, suggesting either lower penetrance of the trait or a possible association with a recessive seed-lethal phenotype. Isoflavone levels were analyzed in individual brown striped and tan seeds from the 1-1 and 1-2 events, as well as brown stripe seeds from the 1-25 event. In every case, the brown striped seeds had high daidzein and low genistein, while the tan seeds had the high genistein control profile (FIG. 13 and FIG. 14, respectively). Thus the brown stripe cosegregates with an altered isoflavone phenotype in seeds obtained from CRC transformants. This visual phenotype provided a means of identifying CRC homozygotes as well as wild type segregants.

Thus, seeds with the high total daidzein to total genistein trait could be identified visually before analysis. Plants that were wild type segregants from the CRC transformation event lines were identified as those plants producing only seeds without the brown stripe and the controls for the field-grown R2 seeds were obtained from these plants.

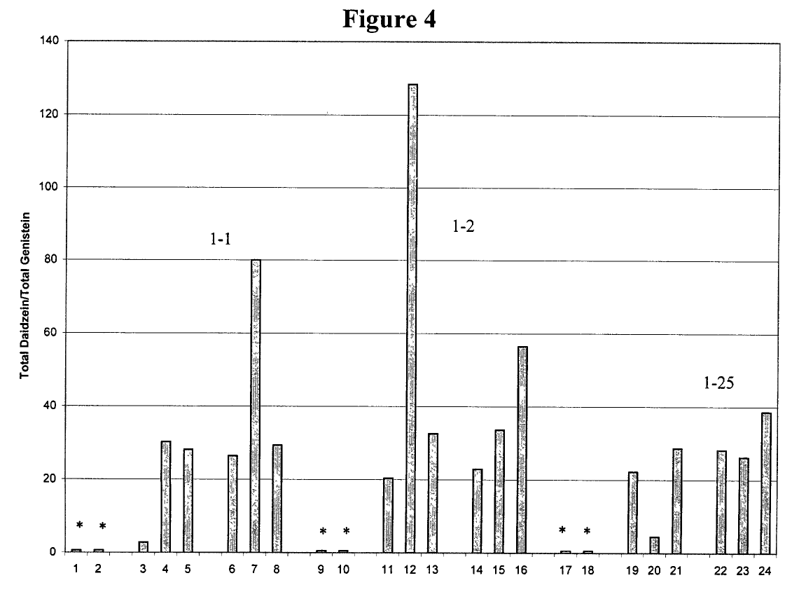

The total daidzein to total genistein ratio for single R2 seeds from field-grown plants: either plants with no seeds with a brown stripe (wild type segregants) or plants with seeds segregating for the brown stripe are shown in FIG. 4. The transgenic plants expressing the CRC recombinant DNA construct were segregating for the phenotype but only data for seeds with a brown stripe are shown. The total daidzein to total genistein ratios in the wild type segregants were between 0.6 and 0.7 while the total daidzein to total genistein ratios in seeds having a brown stripe along the median of the seed ranged between 2.9 and 128.0. Of the 18 seeds having a brown stripe along the median that were analyzed, 16 had total daidzein to total genistein ratios equal to or greater than 20, while the other two seed had ratios of 2.9 and 4.5. Clearly, the high total daidzein to total genistein ratio was inherited in second generation plants of the 1-1, 1-2, and 1-25 events as demonstrated by the isoflavone component levels in the R2 field-grown seeds.

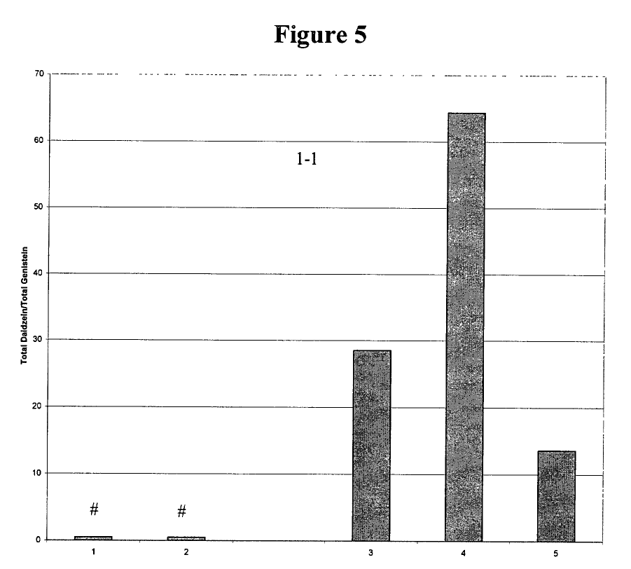

The control seeds for plants grown in the growth room was seed lacking the brown stripe along the median and harvested from the same plant as the seed having the brown stripe along the median of the seed. The total daidzein to total genistein ratios for the R2 seeds from growth room plants was obtained and is shown in FIG. 5. For the R2 seeds from plants of the 1-1 event grown in the growth-room the total daidzein to total genistein ratios were much higher than the ratios for control seeds. The control seeds had total daidzein to total genistein ratios between 0.5 and 0.6 while seeds with the brown stripe down the median had total daidzein to total genistein ratios between 13.6 and 64.4.

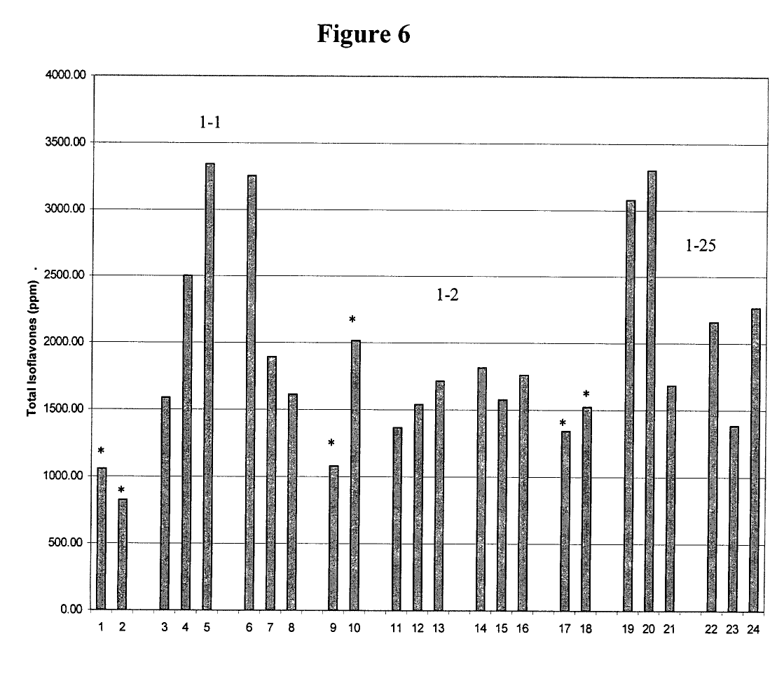

The total isoflavone levels in the R2 seeds from field grown plants were measured and are summarized in FIG. 6. While some seeds containing the brown stripe along the median had total isoflavone levels about two times that of seeds not having the brown stripe, some of this brown stripe seeds had lower total isoflavone levels than the wild type segregant seeds without the brown stripe. Of the seeds from plants resulting from the 1-1 transformation event, all of the seeds with the brown stripe along the median had total isoflavone levels greater than the wild type segregant seeds. Of the seeds from plants resulting from the 1-25 transformation event, the total isoflavone levels of the seeds with the brown stripe along the median were greater than the wild type segregant for all but one of the seeds analyzed. Of the seeds from plants resulting from the 1-2 transformation event, the total isoflavone levels for one of the wild type segregant seeds was higher than the usual control range. The total isoflavone level for this seed was higher than the total isoflavone levels for all of the seed from the 1-2 transformation event having the brown stripe along the median. However, all but one of the seeds from the 1-2 transformation event and having the brown stripe along the median had total isoflavone levels greater than the rest of the wild type segregant seeds (for the 1-1, 1-25, and 1-2 events).

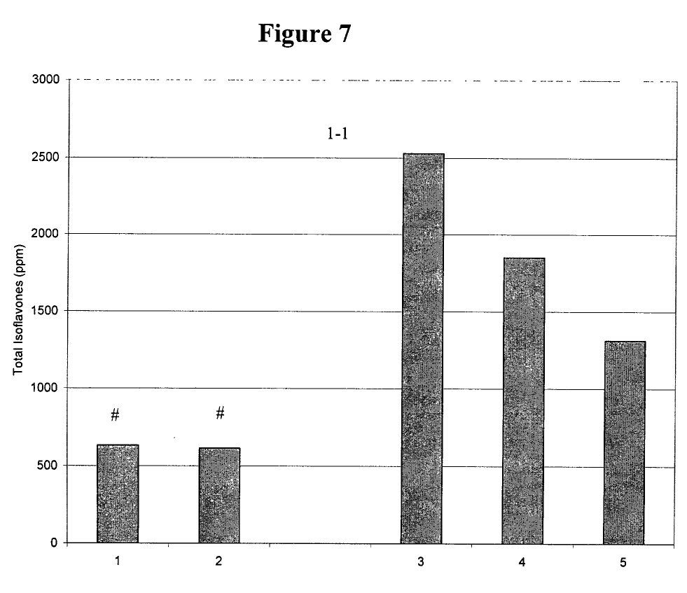

The total isoflavone levels in the R2 seeds from plants of the 1-1 transformation event grown in the growth-room are shown in FIG. 7. Seeds having the brown stripe along the median had higher total isoflavone levels than the control seeds.

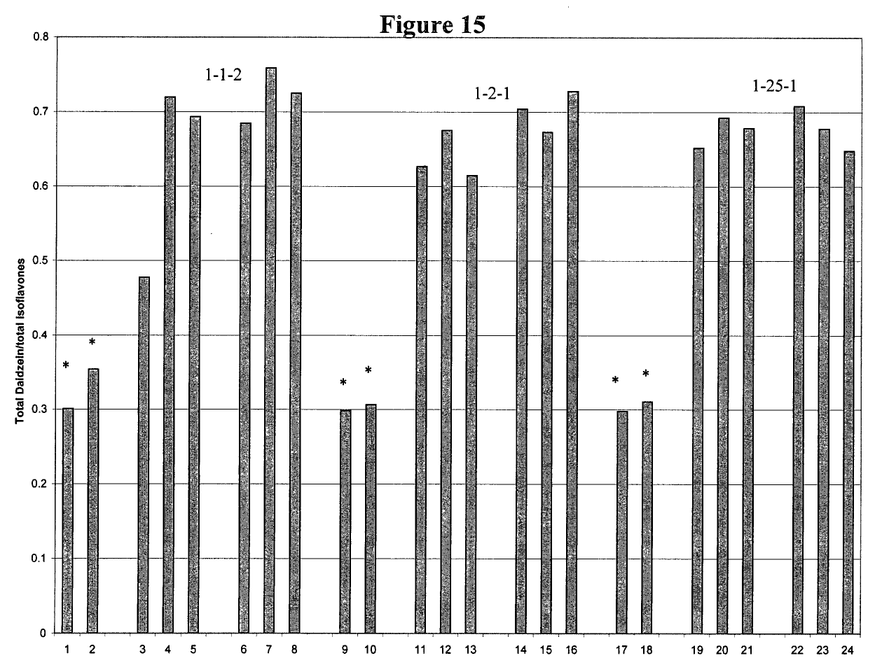

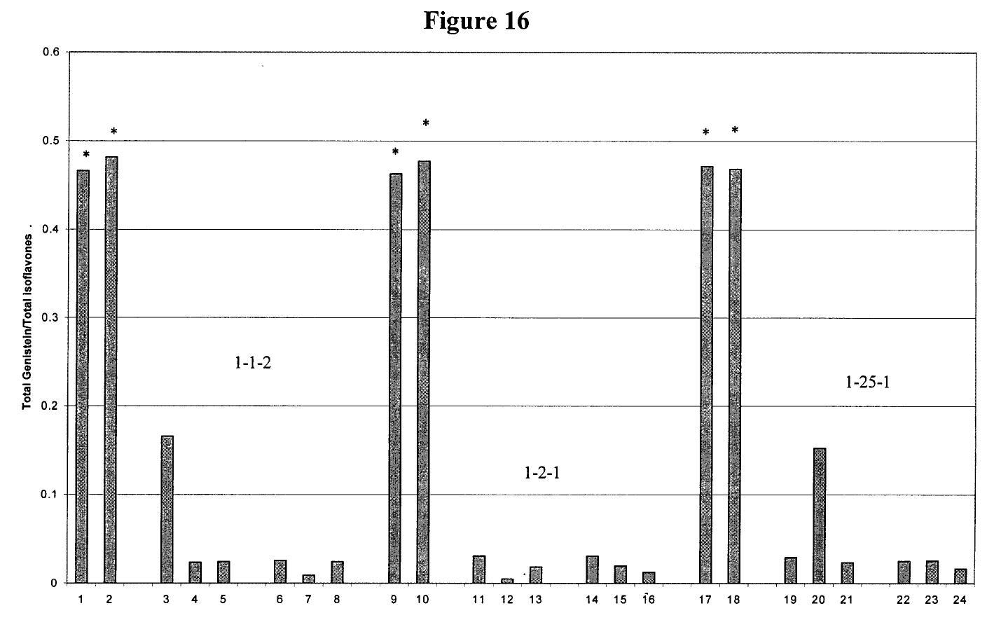

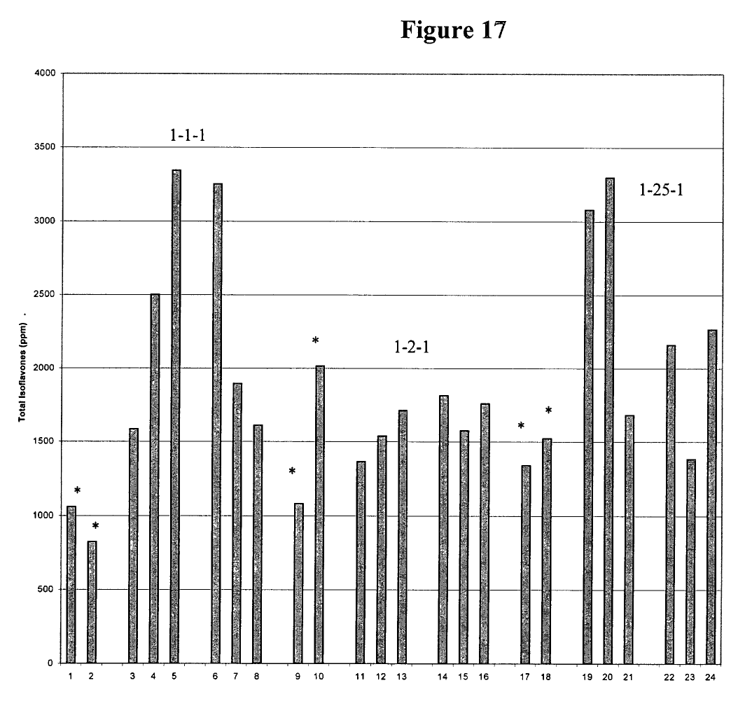

As shown in FIG. 15, the field grown brown striped R2 seeds from all three events had high daidzein levels, and in general had much reduced genistein, with levels around 2%. Even the 1-25 event, which had the least reduced genistein in the R1 seed, showed a greater genistein reduction in field grown seeds (FIG. 16). Thus variations in the extent of genistein reduction occurred between generations and environments. Two individual seeds, one from the 1-1 event and one from the 1-25 event, are notable in having about 15%-17% genistein. This shows that there are also variables that affect the genistein levels even in individual seeds from the same plant. However, overall R2 seeds having the CRC transgene continued to show increased daidzein levels as well as the reduced genistein levels. Also the total isoflavone level was increased in some seeds, but again not consistently (FIG. 17).

In summary, the total isoflavone levels of second generation seeds were higher in most instances for seeds having a brown stripe along the median (indicative of the higher total daidzein to total genistein ratio and of the presence of the CRC recombinant expression construct) than control wild type segregant seeds.

### Example 5

### Analysis of Isoflavones in R3 Seed of Transformants Containing the CRC Recombinant Expression Construct

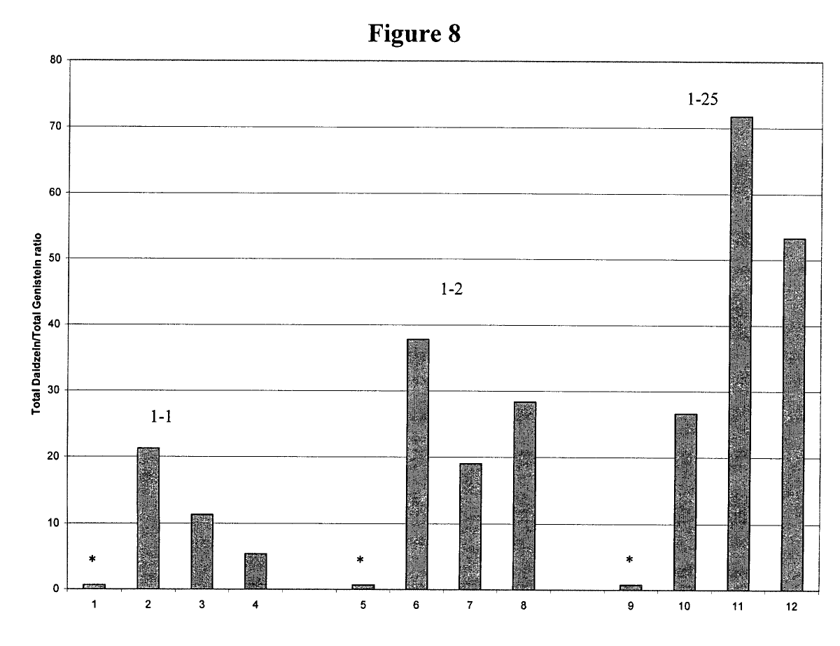

Plants were grown in the growth room from R2 seeds harvested from growth room grown plants from the 1-1, 1-2, and 1-25 transformation events and seeds were harvested (R3) and analyzed for isoflavone content. Extracts were prepared and analyzed in bulk samples as follows. Eight seeds from each plant were combined and ground in a non-commercial grinder. A 200 mg sample was weighed and transferred to a 2 mL vial. The sample was then prepared and assayed as described in Example 4. The controls for this experiment were R3 seeds from wild type segregants producing only non-brown striped seeds. For each transformation event one sample was analyzed from each of one control plant and three plants containing the CRC recombinant expression construct and the results are shown in FIG. 8. The total daidzein to total genistein ratios in the wild type segregant bulk seed samples ranged between 0.7 and 0.8. The total daidzein to total genistein ratios in the samples from plants having the CRC recombinant expression construct ranged between 5.3 and 71.8. Clearly, the high total daidzein to total genistein ratio was inherited in third generation plants of the 1-1, 1-2, and 1-25 events as demonstrated by the isoflavone component levels in the R3 seeds.

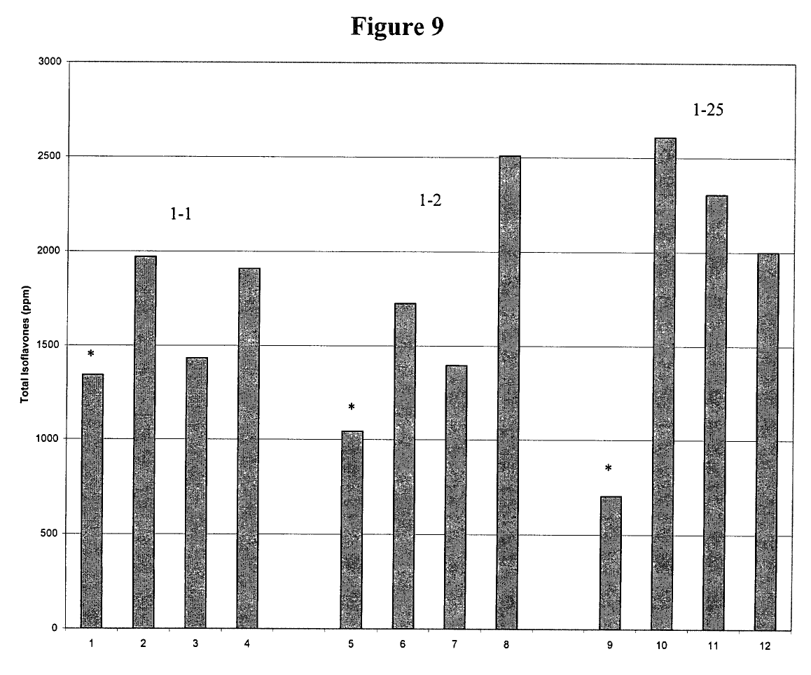

The total isoflavone levels in the bulk R3 samples are shown in FIG. 9. In the R3 seeds, all bulk seed samples from the plants having the CRC recombinant expression construct had total isoflavone levels greater than all of the control plant seed-samples.

### Example 6

### Analysis of the Expression of Genes of the Phenylpropanoid Pathway in R4 Seeds of Transformants Containing the CRC Recombinant Expression Construct

Northern blot and immunoblot analyses were performed to determine the genes in the phenylpropanoid pathway affected by the expression of the CRC recombinant expression construct. Probes were prepared to detect mRNA from phenylalanine ammonia lyase (PAL), cinnamic acid 4-hydroxylase (C4H), chalcone isomerase (CHI), chalcone reductase (CHR), isoflavone synthase (IFS), flavanone 3-hydroxylase (F3H), dihydroflavonol reductase (DFR), flavonol synthase (FS), and isoflavone reductase (IFR). RNA was prepared from seed of R3 plants containing the CRC recombinant expression construct or from controls, transferred to a membrane and hybridized with the probes mentioned above. These Northern blot analyses indicated that the levels of PAL, C4H, CHI, CHR, F3H, DFR, and FS were increased in the seed of transgenic plants expressing the CRC recombinant expression construct compared to controls. Immunoblot analyses were performed on protein samples derived from seed of the same plants, using anti-CHS, anti-CHR, or anti-IFS antisera. The protein expression profiles of CHR and IFS genes correlated with their RNA expression profiles. The CHS protein was increased in seed of CRC transgenic plants, suggesting higher expression of the CHS gene.

### Northern Blot analyses

R3 generation plants of the 1-1 event were grown in the growth chamber. Plants homozygous for the CRC recombinant expression construct, producing only brown-striped seeds, and wild type segregants, producing only tan seeds, were grown. Immature seeds were harvested at two stages of development, at approximately 1 0-days-after flowering and 20-days-after flowering weighing approximately 150 mg and 250 mg, respectively. Total RNA and protein from these materials were extracted separately. For RNA extraction, a modified Trizol method (Gibco BRL, Life Technologies, Rockville, Md.) was applied. Approximately 5 seeds for each sample were ground together in liquid nitrogen and 500 mg of the powder were extracted with 7.5 mL of Trizol reagent for 5 min. Three mL of chloroform was added, mixed, and the 4mL aqueous phase was collected. The RNA was precipitated by the addition of 4 mL of iso-amyl alcohol. After centrifugation and removal of the liquid phase, the RNA precipitate was washed with 75% ethanol and air-dried for 20 min. The RNA was resuspended in 400 μL of water and from each sample, an amount equivalent to 30 μg of RNA was loaded in each lane of a precast Rilant RNA Gel (FMC, Rockland, Me.). The RNA components were separated by electrophoresis and transferred to a membrane following standard protocols for RNA separation and Northern blotting (Sambrook).

Probes were prepared from clones identified in the DuPont EST proprietary database as encoding the desired genes. The sequence of the entire cDNA insert in each chosen clone (except srr1c.pk001.k4) was obtained to verify that the insert represented the correct gene. The clones used to prepare the probes are shown in Table 2 together with the name of the encoded polypeptide and the corresponding identifier (SEQ ID NO:) as used in the attached Sequence Listing.

TABLE 2
Clones Used in the Preparation of Probes for the
Detection of RNA from Genes of the Phenylpropanoid Pathway

| Clone | Encoded Polypeptide | SEQ ID NO: |
|---|---|---|
| sdp3c.pk002.c22 | PAL (phenylalanine ammonia lyase) | 5 |
| src3c.pk014.e17 | C4H (cinnamic acid 4-hydroxylase) | 6 |
| ssm.pk0013.e3* | CHI (chalcone isomerase) | 7 |
| src3c.pk009.e4 | CHR (chalcone reductase) | 8 |
| pOY204* | IFS (isoflavone synthase) | 9 |
| sfl1.pk0040.g11* | F3H (flavanone 3-hydroxylase) | 10 |
| sfl1.pk131.g5** | DFR (dihydroflavonol reductase) | 11 |
| sre.pk0043.d11** | DFR (dihydroflavonol reductase) | 12 |
| ssl.pk0057.d12 | FS (flavonol synthase) | 13 |
| srr1c.pk001.k4 | IFR (isoflavone reductase) | 14 |

*Some of these clones have been described in other patent applications. For example, clone ssm.pk0013.e3 is described in U.S. Pat. No. 6,054,636; clone sfl1.pk0040.g11 is described in PCT publication No. WO 99/43,825, and clone pOY204 is described in PCT publication No. WO 00/44,909.
**Both clones were used together to prepare the probe.

Probes were prepared by the random primer method using the Random Primers DNA Labeling System from GIBCO-BRL, Life Technologies according to the manufacturer's protocol. The entire plasmid was used as template for all probes, except for IFS, where the template was a PCR product containing the IFS coding region. This PCR amplification product was obtained as described in Example 1, above, for the preparation of WSJ001.

The entire random primer reaction mixture, without purification, was used for hybridization. Hybridization conditions were based on a protocol from PerfectHyb Buffer (Sigma-Aldrich, St. Louis, Mo.). Hybridizations were carried out overnight at 68° C. The membranes were then washed twice with 2×SSC buffer (GIBCO BRL, Life Technologies) and once with 0.1×SSC for 15 minutes each at 68° C.

### Immunoblot Analyses

Antibodies to CHS and CHR were prepared by Covance (Richmond, Calif.) to protein purified from E. coli expressing the CHS or CHR coding region using standard methods. The IFS antibody was prepared to synthetic peptides of the IFS protein as described in WO 00/44,909. Standard protocols were used for immunoblot analyses with anti-CHS, anti-CHR, or anti-IFS antisera. The Supersignal West Pico chemiluminescent substrate (Pierce, Rockford, Ill.) was used for visualization of the bound antibodies for CHS and CHR, while the Femto chemiluminescent substrate (Pierce, Rockford, Ill.) was used for IFS.

Table 3 shows the relative detection of the RNA and/or protein of the different genes in the isoflavonoid pathway in either seeds from wild type segregant control 15 plants or seeds from the CRC recombinant expression construct plants, harvested at 150 mg or 250 mg. One plus sign (+) indicates that the RNA or protein is clearly detected; +/− indicates that the RNA or protein is barely detected; and more than one plus sign indicates the approximate increase in detection of the particular RNA or protein levels.

TABLE 3
Levels of Expression of Phenylpropanoid Pathway Genes in
wt Seed and Seed Expressing the CRC Recombinant expression
construct

| | RNA Level | | | | Protein Level | | | |
|---|---|---|---|---|---|---|---|---|
| | 150 mg | | 250 mg | | 150 mg | | 250 mg | |
| Gene | WT | CRC | WT | CRC | WT | CRC | WT | CRC |
| PAL | + | +++++ | + | +++++ | nd* | nd | nd | nd |
| C4H | + | ++++ | + | ++++ | nd | nd | nd | nd |
| CHS | nd | nd | nd | nd | + | +++++ | + | +++++ |
| CHI | + | +++ | + | +++ | nd | nd | nd | nd |
| CHR | + | +++ | + | +++ | + | ++ | + | ++ |
| F3H | + | ++++ | + | ++++ | nd | nd | nd | nd |
| DFR | + | ++++ | +/− | ++++ | nd | nd | nd | nd |
| FS | +/− | +++ | +/− | +++ | nd | nd | nd | nd |
| IFS | ++ | ++ | ++ | ++ | + | + | + | + |
| IFR | +/− | +/− | +/− | + | nd | nd | nd | nd |

*not determined

These results indicate that expression of particular soybean genes of the phenylpropanoid pathway, as listed above, was increased when the CRC recombinant expression construct was expressed in soybean seed. In the upper phenylpropanoid pathway the most dramatic changes were observed in expression of the PAL and CHS genes. Expression of C4H, CHI, and CHR was also increased significantly.

Expression of IFS was not increased. IFR, an enzyme involved in the synthesis of glyceollins from daidzein, was not increased in the younger seed and had a slight increase in the older seed.

Expression of some genes encoding enzymes involved in the flavonol/anthocyanin branch of the phenylpropanoid pathway was increased by CRC expression. These include F3H, DFR, and FS.

It was determined that soybean seed expressing the CRC recombinant expression construct present a brown stripe along the median making them easy to identify. From the analysis of R1, R2, and R3 seed it was determined that the levels of total isoflavones and total daidzein to total genistein ratios vary both in control seed and in seed containing the CRC recombinant expression construct.

Overall the total daidzein to total genistein ratios for seed containing the CRC recombinant expression construct ranged between 2.9 and 801.0 and for samples from control seed ranged between 0.3 and 1.6. There is no overlap in these ranges.

Of the seed examined, the total isoflavone levels were higher in the R1 seed from plants expressing the CRC recombinant expression construct than in plants not expressing the CRC recombinant expression construct. With two exceptions the total isoflavone levels of R2 seed obtained from field-grown plants were higher in seed from plants expressing the CRC recombinant expression construct compared to seed from plants not expressing the recombinant expression construct. In this instance there were two outliers, one seed from the 1-25 transformation event containing the CRC recombinant expression construct had lower total isoflavone levels than seed from the wt-segregants, and one seed from a wt-segregant of the 1-2 transformation event had unusually high total isoflavone levels. All R3 seed examined containing the brown stripe along the median had higher total isoflavone levels than seed from wt-segregants.

### Example 7

### Identification of Intermediates of the Phenylpropanoid Pathway that Accumulate in Transformants Containing the CRC Recombinant expression construct

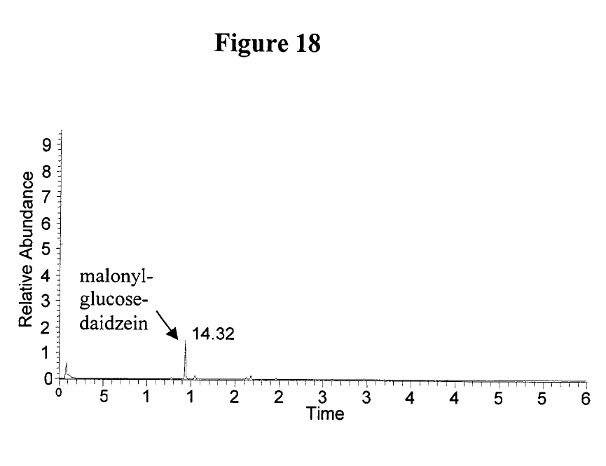

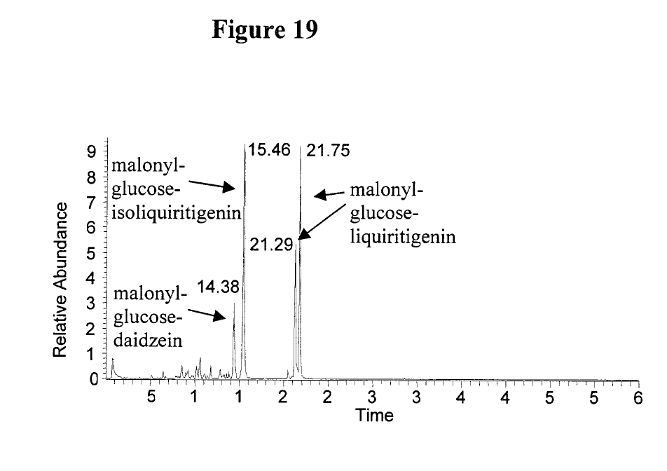

Mass spectroscopy was used to determine the differences in HPLC profiles between soybean seeds expressing the CRC recombinant DNA fragment and control seeds. Using mass spectroscopy three compounds were identified that are almost undetectable in wild type seed but present in seed expressing the CRC recombinant expression construct. Each of the additionally identified compounds has an m/z of 505 but differ in retention times of 15.46, 21.29, and 21.75 min (compare FIG. 18, from wild type seed, and FIG. 19 from seed expressing the CRC recombinant expression construct). MS2 analysis produced one major fragment with an m/z of 257 for each of the compounds. This mass indicates the loss of a fragment with a mass of 248, which is consistent with fragmentation of a malonyl-glucose from a conjugated compound.

Liquiritigenin and isoliquiritigenin, intermediates in daidzein synthesis, both have a mass of 256 matching the m/z of 257 detected for each of the unknown peaks. The unknowns were further analyzed by first using in-source fragmentation (source collision induced dissociation) to remove the 248 m/z fragment leaving the 257 m/z species, followed by MS2. The initial fragmentation was done under conditions determined to be ideal for removal of the malonyl-glucose moiety from the malonyl-glucose derivatives of daidzein and genistein. MS2 produced the same fragments of 239, 147, and 137 for each of the three unknowns. Analysis of liquiritigenin and isoliquiritigenin standards showed MS1 spectra with a major peak of m/z 257 and MS2 fragments of 239,147, and 137 for each compound. These results suggest that the three unknowns are malonyl-glucose derivatives of liquiritigenin and/or isoliquiritigenin.

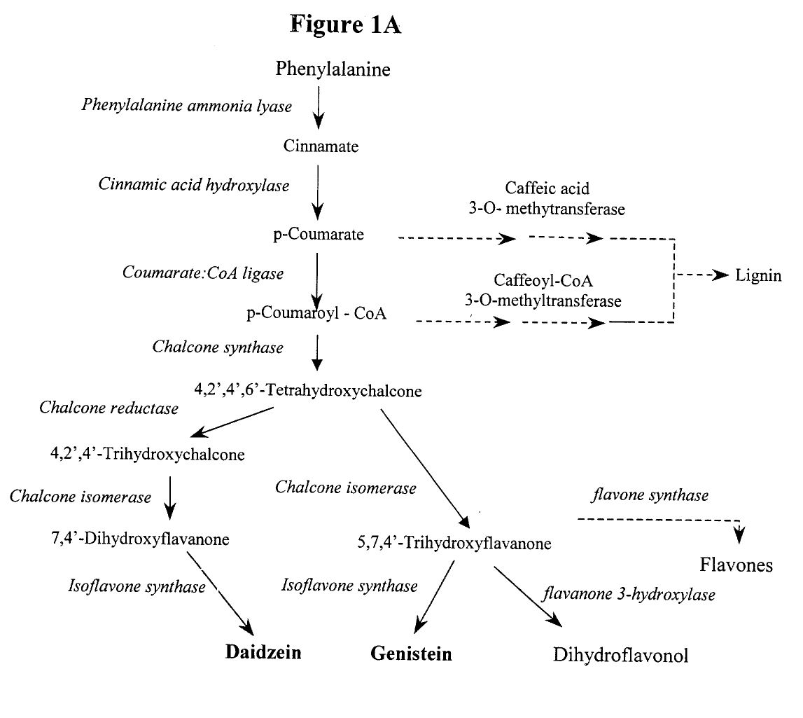

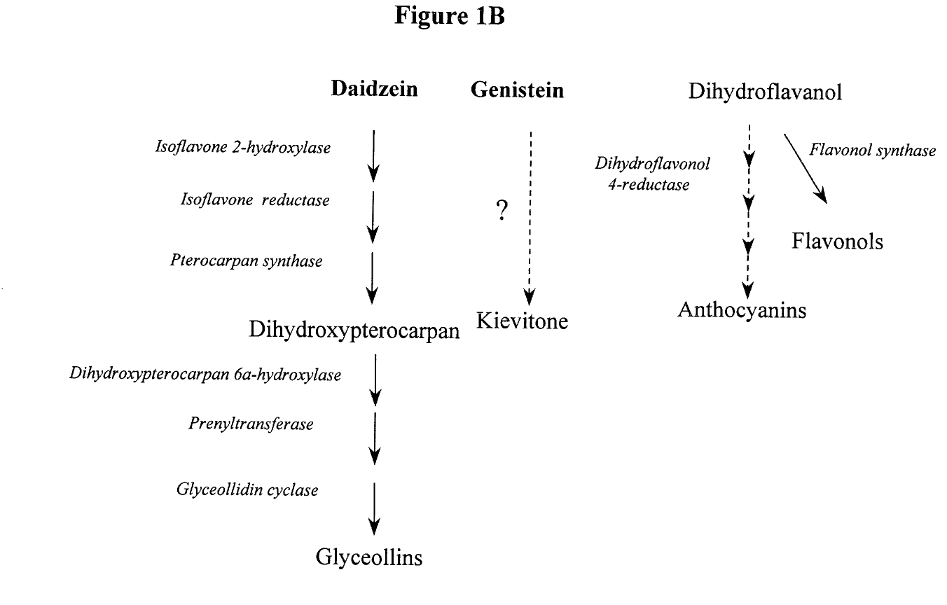

Further characterization of the liquiritigenin and isoliquiritigenin standards showed that the UV spectra and retention times could be used to distinguish the two compounds. The UV spectrum of liquiritigenin matched that of the unknown with the 15.5 retention time, while the spectra of the unknowns at 21.3 and 21.8 both are similar to the isoliquiritigenin UV spectrum (data not shown). The retention times of the unknowns, when compared to the 18.3 and 27.1 retention times of liquiritigenin and isoliquiritigenin, respectively, also match expectations based on the differences between retention times for flavonoid aglycones and their corresponding malonyl-glucose conjugates. From this and the above data, it is concluded that the unknown at 15.5 is the malonyl-glucose conjugate of liquiritigenin, and the unknowns at 21.3 and 21.8 are malonyl-glucose conjugates of isoliquiritigenin. Conjugation of isoliquiritigenin at two different positions probably accounts for the latter two peaks. Accumulation of these intermediates in the CRC seed suggests that the isoflavone synthase catalyzed reaction may be limiting (FIG. 1), although increased capture of intermediates by enhanced activities of genes encoding enzymes involved in conjugation is also a possibility.

What is claimed is:

1. A method of altering the isoflavonoid profile of an isoflavonoid-producing plant, said method comprising:
(a) transforming a plant with (i) a first recombinant expression construct comprising a promoter operably linked to an isolated nucleic acid fragment encoding a C1 myb transcription factor and a recombinant expression construct comprising a promoter operably linked to an isolated nucleic acid fragment encoding an R myc-type transcription factor, (ii) a second recombinant expression construct comprising a promoter operably linked to an isolated nucleic acid fragment encoding a C1 myb transcription factor and a promoter operably linked to an isolated nucleic acid fragment encoding an R myc-type transcription factor, or (iii) a recombinant expression construct comprising a promoter operably linked to an isolated nucleic acid fragment encoding all or part of a C1 myb transcription factor and all or part of an R myc-type wherein said construct is capable of functioning as both a C1 myb transcription factor and an R myc-type transcription factor; and
(b) growing the transformed plant under conditions that are suitable for the expression of the recombinant expression construct or constructs;
wherein expression of the construct or constructs alters the isoflavonoid profile of the transformed plant by increasing the total daidzein to total genistein ratio compared to the total daidzein to total genistein ratio of a control.

2. The method of claim 1 wherein the plant is transformed with a recombinant expression construct comprising a promoter operably linked to an isolated nucleic acid fragment encoding a chimeric transcription factor comprising the maize R coding region situated between the C1 DNA binding domain and the C1 activation domain.

3. The method of claim 1 or claim 2 wherein the promoter is a seed-specific promoter.

4. The method of claim 1 or claim 2 wherein the isoflavonoid-producing plant is selected from the group consisting of soybean, clover, mung bean, lentil, hairy vetch, alfalfa, lupine, sugar beet, and snow pea.

5. An isoflavonoid-producing plant made by the method of claim 1 or 2 wherein said plant has an increased total daidzein to total genistein ratio compared to the total daidzein to total genistein ratio of a control.

6. The isoflavonoid-producing plant of claim 5 wherein said plant is selected from the group consisting of soybean, clover, mung bean, lentil, hairy vetch, alfalfa, lupine, sugar beet, and snow pea.

7. Seeds or plant parts of the plant of claim 5.

8. Seeds or plant parts of the plant of claim 6.

9. An isoflavonoid-containing product having an increased ratio of total daidzein to total genistein obtained from the seeds or plant parts of claim 7.

10. An isoflavonoid-containing product having an increased ratio of total daidzein to total genistein obtained from the seeds or plant parts of claim 8.

11. The isoflavonoid-containing product of claim 9 wherein the isoflavonoid product is selected from the group consisting of protein isolate, protein concentrate, meal, grits, full fat and defatted flours, textured proteins, textured flours, textured concentrates, and textured isolates.

12. The product of claim 10 wherein the isoflavonoid-containing product is selected from the group consisting of protein isolate, protein concentrate, meal, grits, full fat and defatted flours, textured proteins, textured flours, textured concentrates, and textured isolates.

13. An extracted isoflavonoid-containing product having an increased ratio of total daidzein to total genistein wherein said product is extracted from the seeds or plant parts of claim 7.

14. An extracted isoflavonoid-containing product having an increased ratio of total daidzein to total genistein wherein said product is extracted from the seeds or plant parts of claim 8.

15. A food which has incorporated therein the product of claim 9.

16. A food which has incorporated therein the product of claim 10.

17. A beverage which has incorporated therein the product of claim 9.

18. A beverage which has incorporated therein the product of claim 10.

19. An isoflavonoid-containing soy protein product having an increased ratio of total daidzein to total genistein obtained from the seeds of claim 8 wherein the seeds are soybean seeds.

20. The product of claim 19 wherein the isoflavonoid product is selected from the group consisting of protein isolate, protein concentrate, meal, grits, full fat and defatted flours, textured proteins, textured flours, textured concentrates, textured isolates, soymilk, tofu, fermented soy products, and whole bean soy products.

21. An extracted isoflavonoid-containing soy protein product having an increased ratio of total daidzein to total genistein wherein said product is extracted from the seeds of claim 8 wherein the seeds are soybean seeds.

22. A food which has incorporated therein the product of claim 19.

23. A beverage which has incorporated therein the product of claim 19.

24. A method of producing an isoflavonoid-containing product which comprises:
(a) cracking the seeds of claim 7 to remove the meats from the hulls; and
(b) flaking the meats obtained in step (a) to obtain the desired flake thickness.

25. A method of producing an isoflavonoid-containing product which comprises:
(a)cracking the seeds of claim 8 remove the meats from the hulls; and
(b)flaking the meats obtained in step (a) to obtain the desired flake thickness.

26. The method of claim 25 wherein the seeds are soybean seeds.

27. An isoflavonoid-producing plant comprising in its genome
(i) a first recombinant expression construct comprising a promoter operably linked to an isolated nucleic acid fragment encoding a C1 myb transcription factor and a recombinant expression construct comprising a promoter operably linked to an isolated nucleic acid fragment encoding an R myc-type transcription factor,
(ii) a second recombinant expression construct comprising a promoter operably linked to an isolated nucleic acid fragment encoding a C1 myb transcription factor and a promoter operably linked to an isolated nucleic acid fragment encoding an R myc-type transcription factor, or
(iii) a recombinant expression construct comprising a promoter operably linked to an isolated nucleic acid fragment encoding all or part of a C1 myb transcription factor and all or part of an R myc-type wherein said construct is capable of functioning as both a C1 myb transcription factor and an R myc-type transcription factor;
wherein said plant has an increased total daidzein to total genistein ratio when compared to the total daidzein to total genistein ratio of a control.

28. The isoflavonoid-producing plant of claim 27 wherein the recombinant expression construct (iii) comprises a promoter operably linked to an isolated nucleic acid fragment encoding a chimeric transcription factor comprising the maize R coding region situated between the C1 DNA binding domain and the C1 activation domain.

29. The isoflavonoid-producing plant of claim 27 or 28 wherein the promoter is a seed-specific promoter.

30. The isoflavonoid-producing plant of claim 27 or claim 28 wherein the isoflavonoid-producing plant is selected from the group consisting of soybean, clover, mung bean, lentil, hairy vetch, alfalfa, lupine, sugar beet, and snow pea.

31. The isoflavonoid-producing plant of claim 27 or 28 wherein said plant has an increased total daidzein to total genistein ratio compared to the total daidzein to total genistein ratio of a control.

32. The isoflavonoid-producing plant of claim 31 wherein said plant is selected from the group consisting of soybean, clover, mung bean, lentil, hairy vetch, alfalfa, lupine, sugar beet, and snow pea.

33. Seeds or plant parts of the plant of claim 31.

34. Seeds or plant parts of the plant of claim 32.

35. An isoflavonoid-containing product having an increased ratio of total daidzein to total genistein obtained from the seeds or plants parts of claim 33.

36. An isoflavonoid-containing product having an increased ratio of total daidzein to total genistein obtained from the seeds or plant parts of claim 34.

37. The product of claim 35 wherein the isoflavonoid product is selected from the group consisting of protein isolate, protein concentrate, meal, grits, full fat and defatted flours, textured proteins, textured flours, textured concentrates, and textured isolates.

38. The product of claim 36 wherein the isoflavonoid-containing product is selected from the group consisting of protein isolate, protein concentrate, meal, grits, full fat and defatted flours, textured proteins, textured flours, textured concentrates, and textured isolates.

39. An extracted isoflavonoid-containing product having an increased ratio of total daidzein to total genistein wherein said product is extracted from the seeds or plant parts of claim 33.

40. An extracted isoflavonoid-containing product having an increased ratio of total daidzein to total genistein wherein said product is extracted from the seeds or plant parts of claim 34.

41. A food which has incorporated therein the product of claim 35.

42. A food which has incorporated therein the product of claim 36.

43. A beverage which has incorporated therein the product of claim 35.

44. A beverage which has incorporated therein the product of claim 36.

45. An isoflavonoid-containing soy protein product having an increased ratio of total daidzein to total genistein obtained from the seeds of claim 34 wherein the seeds are soybean seeds.

46. The product of claim 44 wherein the isoflavonoid product is selected from the group consisting of protein isolate, protein concentrate, meal, grits, full fat and defatted flours, textured proteins, textured flours, textured concentrates, textured isolates, soymilk, tofu, fermented soy products, and whole bean soy products.

47. An extracted isoflavonoid-containing soy protein product having an increased ratio of total daidzein to total genistein wherein said product is extracted from the seeds of claim 34 wherein the seeds are soybean seeds.

48. A food which has incorporated therein the product of claim 40.

49. A beverage which has incorporated therein the product of claim 40.

50. A method of producing an isoflavonoid-containing product which comprises:
(a) cracking the seeds of claim 33 to remove the meats from the hulls; and
(b) flaking the meats obtained in step (a) to obtain the desired flake thickness.

51. A method of producing an isoflavonoid-containing product which comprises:
(a) cracking the seeds of claim 34 remove the meats from the hulls; and
(b) flaking the meats obtained in step (a) to obtain the desired flake thickness.

52. The method of claim 50 wherein the seeds are soybean seeds.
